SEQUENCE LISTING (31 sequences)

SEQ ID NO: 1 - DNA, 597 bp, Artificial Sequence
  socs2 (suppressor of cytokine signaling 2) cDNA
atgaccctgcggtgccttgagccctccgggaatggcggggaagggacgcggagccagtgggggaccgcggggtcggcggaggagccatccccgcaggcggcgcgtctggcgaaggccctgcgggagctcggtcagacaggatggtactggggaagtatgactgttaatgaagccaaagagaaattaaaagaggcaccagaaggaactttcttgattagagatagctcgcattcagactacctactaacaatatctgttaaaacatcagctggaccaactaatcttcgaatcgaataccaagacggaaaattcagattggactctatcatatgtgtcaaatccaagcttaaacaatttgacagtgtggttcatctgatcgactactatgttcagatgtgcaaggataagcggacaggtccagaagccccccggaacggcactgttcacctttatctgaccaaaccgctctacacgtcagcaccatctctgcagcatctctgtaggctcaccattaacaaatgtaccggtgccatctggggactgcctttaccaacaagactaaaagattacttggaagaatataaattccaggtataa

SEQ ID NO: 2 - RNA, 58 nt, Artificial Sequence
  shRNA for socs2(suppressor of cytokine signaling 2)
ccggcgcauucagacuaccuacuaacucgaguuaguagguagucugaaugcguuuuug

SEQ ID NO: 3 - RNA, 57 nt, Artificial Sequence
  negative control shRNA for socs2(suppressor of cytokine signaling 2)
ccggcaacaagaugaagagcaccaacucgaguuggugcucuucaucuuguuguuuuu

SEQ ID NO: 4 - DNA, 22 bp, Artificial Sequence
  socs2(suppressor of cytokine signaling 2) sense primer for PCR
taaaagaggcaccagaaggaac

SEQ ID NO: 5 - DNA, 21 bp, Artificial Sequence
  socs2(suppressor of cytokine signaling 2) antisense primer for PCR
tcgatcagatgaaccacactg

SEQ ID NO: 6 - DNA, 20 bp, Artificial Sequence
  Sense primer for PCR of GAPDH as quantitative control
cagcctcaagatcatcagca

SEQ ID NO: 7 - DNA, 20 bp, Artificial Sequence
  antisense primer for PCR of GAPDH as quantitative control
gtcttctgggtggcagtgat

SEQ ID NO: 8 - DNA, 20 bp, Artificial Sequence
  socs1(suppressor of cytokine signaling 1) sense primer for PCR
agagcttcgactgcctcttc

SEQ ID NO: 9 - DNA, 20 bp, Artificial Sequence
  socs1(suppressor of cytokine signaling 1) antisense primer for PCR
ctcaggtagtcgcggaggac

SEQ ID NO: 10 - DNA, 20 bp, Artificial Sequence
  socs3(suppressor of cytokine signaling 3) sense primer for PCR
gccacctactgaaccctcct

SEQ ID NO: 11 - DNA, 20 bp, Artificial Sequence
  socs3(suppressor of cytokine signaling 3) antisense primer for PCR
acggtcttccgacagagatg

SEQ ID NO: 12 - RNA, 19 nt, Artificial Sequence
  socs2(suppressor of cytokine signaling 2) siRNA for inhibiting socs2 expression
cgacuacuauguucagaug

SEQ ID NO: 13 - RNA, 21 nt, Artificial Sequence
  control siRNA for inhibiting socs2 expression
uagcgacuaaacacaucaauu

SEQ ID NO: 14 - DNA, 20 bp, Artificial Sequence
  IFN gamma sense primer for real time PCR
gtccaacgcaaagcaataca

SEQ ID NO: 15 - DNA, 20 bp, Artificial Sequence
  IFN gamma antisense primer for real time PCR
ctcttcgacctcgaaacagc

SEQ ID NO: 16 - DNA, 46 bp, Artificial Sequence
  forward primer for PCR amplification of the socs2 gene
ggatccatgaccctgcggtgccttgagccctccgggaatggcgggg

SEQ ID NO: 17 - DNA, 47 bp, Artificial Sequence
  reverse primer for PCR amplification of the socs2 gene
atcgatttatacctggaatttatattcttccaagtaatcttttagtc

SEQ ID NO: 18 - DNA, 3030 bp, Artificial Sequence
  The cDNA encoding Pyk2 (protein tyrosine kinase 2)
atgtctggggtgtccgagcccctgagtcgagtaaagttgggcacgttacgccggcctgaaggccctgcagagcccatggtggtggtaccagtagatgtggaaaaggaggacgtgcgtatcctcaaggtctgcttctatagcaacagcttcaatcctgggaaaaacttcaaactggtcaaatgcactgtccagacggagatccgggagatcatcacctccatcctgctgagcgggcggatcgggcccaacatccggttggctgagtgctatgggctgaggctgaagcacatgaagtccgatgagatccactggctgcacccacagatgacggtgggtgaggtgcaggacaagtatgagtgtctgcacgtggaagccgagtggaggtatgaccttcaaatccgctacttgccagaagacttcatggagagcctgaaggaggacaggaccacgctgctctatttttaccaacagctccggaacgactacatgcagcgctacgccagcaaggtcagcgagggcatggccctgcagctgggctgcctggagctcaggcggttcttcaaggatatgccccacaatgcacttgacaagaagtccaacttcgagctcctagaaaaggaagtggggctggacttgtttttcccaaagcagatgcaggagaacttaaagcccaaacagttccggaagatgatccagcagaccttccagcagtacgcctcgctcagggaggaggagtgcgtcatgaagttcttcaacactctcgccggcttcgccaacatcgaccaggagacctaccgctgtgaactcattcaaggatggaacattactgtggacctggtcattggccctaaagggatccgccagctgactagtcaggacgcaaagcccacctgcctggccgagttcaagcagatcaggtccatcaggtgcctcccgctggaggagggccaggcagtacttcagctgggcattgaaggtgccccccaggccttgtccatcaaaacctcatccctagcagaggctgagaacatggctgacctcatagacggctactgccggctgcagggtgagcaccaaggctctctcatcatccatcctaggaaagatggtgagaagcggaacagcctgccccagatccccatgctaaacctggaggcccggcggtcccacctctcagagagctgcagcatagagtcagacatctacgcagagattcccgacgaaaccctgcgaaggcccggaggtccacagtatggcattgcccgtgaagatgtggtcctgaatcgtattcttggggaaggcttttttggggaggtctatgaaggtgtctacacaaatcacaaaggggagaaaatcaatgtagctgtcaagacctgcaagaaagactgcactctggacaacaaggagaagttcatgagcgaggcagtgatcatgaagaacctcgaccacccgcacatcgtgaagctgatcggcatcattgaagaggagcccacctggatcatcatggaattgtatccctatggggagctgggccactacctggagcggaacaagaactccctgaaggtgctcaccctcgtgctgtactcactgcagatatgcaaagccatggcctacctggagagcatcaactgcgtgcacagggacattgctgtccggaacatcctggtggcctcccctgagtgtgtgaagctgggggactttggtctttcccggtacattgaggacgaggactattacaaagcctctgtgactcgtctccccatcaaatggatgtccccagagtccattaacttccgacgcttcacgacagccagtgacgtctggatgttcgccgtgtgcatgtgggagatcctgagctttgggaagcagcccttcttctggctggagaacaaggatgtcatcggggtgctggagaaaggagaccggctgcccaagcctgatctctgtccaccggtcctttataccctcatgacccgctgctgggactacgaccccagtgaccggccccgcttcaccgagctggtgtgcagcctcagtgacgtttatcagatggagaaggacattgccatggagcaagagaggaatgctcgctaccgaacccccaaaatcttggagcccacagccttccaggaacccccacccaagcccagccgacctaagtacagaccccctccgcaaaccaacctcctggctccaaagctgcagttccaggttcctgagggtctgtgtgccagctctcctacgctcaccagccctatggagtatccatctcccgttaactcactgcacaccccacctctccaccggcacaatgtcttcaaacgccacagcatgcgggaggaggacttcatccaacccagcagccgagaagaggcccagcagctgtgggaggctgaaaaggtcaaaatgcggcaaatcctggacaaacagcagaagcagatggtggaggactaccagtggctcaggcaggaggagaagtccctggaccccatggtttatatgaatgataagtccccattgacgccagagaaggaggtcggctacctggagttcacagggcccccacagaagcccccgaggctgggcgcacagtccatccagcccacagctaacctggaccggactgatgacctggtgtacctcaatgtcatggagctggtgcgggccgtgctggagctcaagaatgagctctgtcagctgccccccgagggctacgtggtggtggtgaagaatgtggggctgaccctgcggaagctcatcgggagcgtggatgatctcctgccttccttgccgtcatcttcacggacagagatcgagggcacccagaaactgctcaacaaagacctggcagagctcatcaacaagatgcggctggcacagcagaacgccgtgacctccctaagtgaggagtgcaagaggcagatgctgacggcttcacacaccctggctgtggacgccaagaacctgctcgacgctgtggaccaggccaaggttctggccaatctggcccacccacctgcagagtga

SEQ ID NO: 19 - DNA, 48 bp, Artificial Sequence
  forward primer for PCR amplification of the Pyk2 gene
gaattcgatgtctggggtgtccgagcccctgagtcgagtaaagttggg

SEQ ID NO: 20 - DNA, 48 bp, Artificial Sequence
  reverse primer for PCR amplification of the Pyk2 gene
gtcgactcactctgcaggtgggtgggccagattggccagaaccttggc

SEQ ID NO: 21 - DNA, 315 bp, Artificial Sequence
  SH2 (Src homology 2) domain sequence
acaggatggtactggggaagtatgactgttaatgaagccaaagagaaattaaaagaggcaccagaaggaactttcttgattagagatagctcgcattcagactacctactaacaatatctgttaaaacatcagctggaccaactaatcttcgaatcgaataccaagacggaaaattcagattggactctatcatatgtgtcaaatccaagcttaaacaatttgacagtgtggttcatctgatcgactactatgttcagatgtgcaaggataagcggacaggtccagaagccccccggaacggcactgttcacctt

SEQ ID NO: 22 - DNA, 135 bp, Artificial Sequence
  mutant socs2 in which SH2 (Src homology 2) domain was deleted
atgaccctgcggtgccttgagccctccgggaatggcggggaagggacgcggagccagtgggggaccgcggggtcggcggaggagccatccccgcaggcggcgcgtctggcgaaggccctgcgggagctcggtcag

SEQ ID NO: 23 - DNA, 46 bp, Artificial Sequence; identical to SEQ ID NO: 16
  forward primer for PCR amplification of the mutant socs2 (SEQ ID NO22)

SEQ ID NO: 24 - DNA, 44 bp, Artificial Sequence
  reverse primer for PCR amplification of the mutant socs2 (SEQ ID NO22)
atcgatttactgaccgagctcccgcagggccttcgccagacgcg

SEQ ID NO: 25 - DNA, 450 bp, Artificial Sequence
  the mutant socs2 in which SOCS domain was deleted
atgaccctgcggtgccttgagccctccgggaatggcggggaagggacgcggagccagtgggggaccgcggggtcggcggaggagccatccccgcaggcggcgcgtctggcgaaggccctgcgggagctcggtcagacaggatggtactggggaagtatgactgttaatgaagccaaagagaaattaaaagaggcaccagaaggaactttcttgattagagatagctcgcattcagactacctactaacaatatctgttaaaacatcagctggaccaactaatcttcgaatcgaataccaagacggaaaattcagattggactctatcatatgtgtcaaatccaagcttaaacaatttgacagtgtggttcatctgatcgactactatgttcagatgtgcaaggataagcggacaggtccagaagccccccggaacggcactgttcacctt

SEQ ID NO: 26 - DNA, 46 bp, Artificial Sequence; identical to SEQ ID NO: 16
  forward primer for PCR amplification of the mutant socs2 in which SOCS domain was deleted

SEQ ID NO: 27 - DNA, 45 bp, Artificial Sequence
  reverse primer for PCR amplification of the mutant socs2 in which SOCS domain was deleted
atcgatttaaaggtgaacagtgccgttccggggggcttctggacc

SEQ ID NO: 28 - DNA, 3030 bp, Artificial Sequence
  mutant Pyk2 cDNA sequence
atgtctggggtgtccgagcccctgagtcgagtaaagttgggcacgttacgccggcctgaaggccctgcagagcccatggtggtggtaccagtagatgtggaaaaggaggacgtgcgtatcctcaaggtctgcttctatagcaacagcttcaatcctgggaaaaacttcaaactggtcaaatgcactgtccagacggagatccgggagatcatcacctccatcctgctgagcgggcggatcgggcccaacatccggttggctgagtgctatgggctgaggctgaagcacatgaagtccgatgagatccactggctgcacccacagatgacggtgggtgaggtgcaggacaagtatgagtgtctgcacgtggaagccgagtggaggtatgaccttcaaatccgctacttgccagaagacttcatggagagcctgaaggaggacaggaccacgctgctctatttttaccaacagctccggaacgactacatgcagcgctacgccagcaaggtcagcgagggcatggccctgcagctgggctgcctggagctcaggcggttcttcaaggatatgccccacaatgcacttgacaagaagtccaacttcgagctcctagaaaaggaagtggggctggacttgtttttcccaaagcagatgcaggagaacttaaagcccaaacagttccggaagatgatccagcagaccttccagcagtacgcctcgctcagggaggaggagtgcgtcatgaagttcttcaacactctcgccggcttcgccaacatcgaccaggagacctaccgctgtgaactcattcaaggatggaacattactgtggacctggtcattggccctaaagggatccgccagctgactagtcaggacgcaaagcccacctgcctggccgagttcaagcagatcaggtccatcaggtgcctcccgctggaggagggccaggcagtacttcagctgggcattgaaggtgccccccaggccttgtccatcaaaacctcatccctagcagaggctgagaacatggctgacctcatagacggctactgccggctgcagggtgagcaccaaggctctctcatcatccatcctaggaaagatggtgagaagcggaacagcctgccccagatccccatgctaaacctggaggcccggcggtcccacctctcagagagctgcagcatagagtcagacatcttcgcagagattcccgacgaaaccctgcgaaggcccggaggtccacagtatggcattgcccgtgaagatgtggtcctgaatcgtattcttggggaaggcttttttggggaggtctatgaaggtgtctacacaaatcacaaaggggagaaaatcaatgtagctgtcaagacctgcaagaaagactgcactctggacaacaaggagaagttcatgagcgaggcagtgatcatgaagaacctcgaccacccgcacatcgtgaagctgatcggcatcattgaagaggagcccacctggatcatcatggaattgtatccctatggggagctgggccactacctggagcggaacaagaactccctgaaggtgctcaccctcgtgctgtactcactgcagatatgcaaagccatggcctacctggagagcatcaactgcgtgcacagggacattgctgtccggaacatcctggtggcctcccctgagtgtgtgaagctgggggactttggtctttcccggtacattgaggacgaggactattacaaagcctctgtgactcgtctccccatcaaatggatgtccccagagtccattaacttccgacgcttcacgacagccagtgacgtctggatgttcgccgtgtgcatgtgggagatcctgagctttgggaagcagcccttcttctggctggagaacaaggatgtcatcggggtgctggagaaaggagaccggctgcccaagcctgatctctgtccaccggtcctttataccctcatgacccgctgctgggactacgaccccagtgaccggccccgcttcaccgagctggtgtgcagcctcagtgacgtttatcagatggagaaggacattgccatggagcaagagaggaatgctcgctaccgaacccccaaaatcttggagcccacagccttccaggaacccccacccaagcccagccgacctaagtacagaccccctccgcaaaccaacctcctggctccaaagctgcagttccaggttcctgagggtctgtgtgccagctctcctacgctcaccagccctatggagtatccatctcccgttaactcactgcacaccccacctctccaccggcacaatgtcttcaaacgccacagcatgcgggaggaggacttcatccaacccagcagccgagaagaggcccagcagctgtgggaggctgaaaaggtcaaaatgcggcaaatcctggacaaacagcagaagcagatggtggaggactaccagtggctcaggcaggaggagaagtccctggaccccatggtttatatgaatgataagtccccattgacgccagagaaggaggtcggctacctggagttcacagggcccccacagaagcccccgaggctgggcgcacagtccatccagcccacagctaacctggaccggactgatgacctggtgtacctcaatgtcatggagctggtgcgggccgtgctggagctcaagaatgagctctgtcagctgccccccgagggctacgtggtggtggtgaagaatgtggggctgaccctgcggaagctcatcgggagcgtggatgatctcctgccttccttgccgtcatcttcacggacagagatcgagggcacccagaaactgctcaacaaagacctggcagagctcatcaacaagatgcggctggcacagcagaacgccgtgacctccctaagtgaggagtgcaagaggcagatgctgacggcttcacacaccctggctgtggacgccaagaacctgctcgacgctgtggaccaggccaaggttctggccaatctggcccacccacctgcagagtga

SEQ ID NO: 29 - DNA, 42 bp, Artificial Sequence
  mutant Pyk2 mutation primer
cagcatagagtcagacatcttcgcagagattcccgacgaaac

SEQ ID NO: 30 - DNA, 47 bp, Artificial Sequence
  Pyk2 GFP sense primer
ctcgagccatgtctggggtgtccgagcccctgagtcgagtaaagttg

SEQ ID NO: 31 - DNA, 48 bp, Artificial Sequence
  Pyk2 GFP antisense primer
gaattctcactctgcaggtgggtgggccagattggccagaaccttggc

(19) United States
(12) Patent Application Publication
(10) Pub. No.: US 2012/0282646 A1
(43) Pub. Date: Nov. 8, 2012

(54) METHOD FOR ACTIVATING A NATURAL KILLER CELL BY ADJUSTING THE EXPRESSION OF THE SOCS2 GENE

(75) Inventors: Inpyo Choi, Daejeon (KR); Suk Hyung Lee, Daejeon (KR); Suk-Ran Yoon, Daejeon (KR)

(73) Assignee: Korea Research Institute of Bioscience and Biotechnology

(21) Appl. No.: 13/504,109

(22) PCT Filed: Aug. 30, 2010
(86) PCT No.: PCT/KR2010/005834

§ 371 Date: Apr. 25, 2012

(30) Foreign Application Priority Data
Oct. 26, 2009 (KR) 10-2009-0101784

(51) Int. Cl. C12N 5/0783; C12Q 1/02; C12N 15/85

(52) U.S. Cl. 435/29; 435/375; 435/455

(57) ABSTRACT

The present invention relates to a method for activating natural killer cells (NK cells), and more particularly, to a method for enhancing the cytotoxicity of natural killer cells by inducing the overexpression of suppressor of cytokine signaling 2 (SOCS2) which is a protein involved in cell-signaling pathways in natural killer cells. The inventors of the present invention observed that when natural killer cells were treated with IL-15, a cytokine involved in natural killer cell differentiation, the expression of SOCS2 increased and the expression of proline-rich tyrosine kinase 2 (Pyk2) was inhibited by the SOCS2, the expression of which increased. In addition, when Pyk2 was overexpressed, the ability to produce IFN-γ and the ability to kill tumor cells of natural killer cells decreased. Therefore, SOCS2 can be used for activating natural killer cells and the natural killer cells activated by the method can be used for the prevention or treatment of cancer.

### CROSS-REFERENCES TO RELATED APPLICATION

This patent application claims the benefit of priority under 35 U.S.C. §119 from Korean Patent Application Nos. 10-2009-0101784 filed on Oct. 26, 2009 and PCT Patent Application No. PCT/KR2010/005834 filed on Aug. 30, 2010, the contents of which are incorporated herein by reference.

### BACKGROUND OF THE INVENTION

1. Field of the Invention

The present disclosure relates to a method for activating natural killer cells.

2. Description of the Related Art

Natural killer cells (NK cells) are lymphocytes that are capable of killing tumor cells or virus-infected cells and play an important role in innate immunity (G Trinchieri, Adv Immunol., 47:187-376, 1989). The major mechanism used by NK cells to destroy target cells is the secretion of lytic granules such as perforin and granzyme B through the immune synapse into target cells. Secreted perforin creates pores in the target cell membrane and granzyme B which is allowed to move into the target cells induces caspase-dependent or caspase-independent apoptosis I (Voskoboinik et al., Nat Rev Immunol., 6:940-952, 2006). In addition, NK cells have the ability to produce and secrete IFN-γ. IFN-γ is a cytokine that plays an important role in activating macrophages, linking the innate and adaptive immune responses, and suppressing the proliferation of tumor cells and virus-infected cells (CA Biron et al., Annu Rev Immunol., 17:189-220, 1999). Prior to meeting the target cells, NK cells need priming to have these abilities. For that reason, the ability to kill tumor cells and the ability to produce IFN-γ have been significantly reduced in primary NK cells isolated from humans and mice. It has been reported that examples of NK cell-priming cytokines which can maximize the abilities of NK cells are IL-2 and IL-15, and IL-15 is an essential cytokine for NK cell activity (M Lucas et al., Immunity, 26:503-517, 2007).

SOCS2 is a member of the suppressor of cytokine signaling (SOCS) family and has a Src homology 2 (SH2) domain and a SOCS box. SOCS family proteins have been reported to combine with proteins which play important roles in cellular signaling pathways to block further signal transduction or allow the ubiquitin-mediated proteasomal degradation of combined proteins (A Yoshimura et al., Nat Rev Immunol., 7:454-465, 2007). Particularly, SOCS2 has been shown to regulate the growth hormone, insulin growth factor I, and prolactin signaling pathways. Recently, SOCS2 has been reported to involve in ubiquitin-mediated proteasomal degradation of tumor necrosis factor (TNF) receptor-associated factors (TRAF) 2 and 6 in dendritic cells (Fabiana S. Machado et al., J Exp Med., 205:1077-1086, 2008). However, there have been no reports about the role of SOCS2 in other immune cells, particularly in NK cells.

Proline-rich tyrosine kinase 2 (Pyk2) is a member of the focal adhesion kinase (FAK) non-receptor tyrosine kinase family and has been known to be expressed in neural and hematopoietic stem cells (Avraham et al., J. Biol. Chem., 270:27742-27751, 1995). Pyk2 has been reported to be activated by a variety of stimuli, especially by stimuli that elevate the concentration of intracellular calcium ion (Lev et al., Nature., 376:737745, 1995). In addition, PyK2 interacts with Src kinase (sarcoma, proto-oncogenic tyrosine kinases) and plays a role in mediating heterotrimeric G-protein-coupled receptor and mitogen-activated protein (MAP) kinase signal transduction pathway (Dikic et al., Nature., 383:547550, 1996). Interestingly, overexpressed Pyk2 has been reported to reduce the ability of NK cells to kill tumor cells (Sancho et al., J Cell Biology., 149:1249-1261, 2000). However, there have been no reports about the precise mechanism of regulating Pyk2 in NK cells so far.

Thus, the present inventors found that the expression of SOCS2 increases during IL-15-mediated NK cell priming and the increased SOCS2 maintains NK cell activity via control of phosphorylated Pyk2, and identified that SOCS2 can be used for a pharmaceutical composition for activating NK cells, thereby leading to completion of the present invention.

### SUMMARY OF THE INVENTION

One object of the present invention is to provide a pharmaceutical composition for activating natural killer (NK) cells, comprising an expression vector wherein the socs2 (suppressor of cytokine signaling 2) gene having polynucleotide sequence of SEQ ID NO:1 is operably linked or SOCS2 protein encoded by the socs2 gene as an active ingredient.

Another object of the present invention is to provide a method for activating NK cells comprising treating NK cells in vitro with SOCS2 protein comprising an SH2 (Src homology 2) domain encoded by a nucleic acid molecule having polynucleotide sequence of SEQ ID NO:21, and NK cells activated by the method.

Still another object of the present invention is to provide a method for activating NK cells comprising the steps of:

(1) preparing an expression vector wherein the socs2 gene having polynucleotide sequence of SEQ ID NO:1 is operably linked; and

(2) transducing the expression vector prepared in step (1) into NK cells,

and NK cells activated by the method.

Even another object of the present invention is to provide a method for screening a mutant SOCS2 having the increased NK cell-activating effect, comprising the steps of:

(1) preparing a first expression vector wherein the Pyk2 gene having polynucleotide sequence of SEQ ID NO:18 is operably linked;

(2) preparing second expression vectors wherein a polynucleotide is operably linked, the polynucleotide encoding a mutant SOCS2 in which a SH2 domain encoded by the polynucleotide of SEQ ID NO:21 is conserved within SOCS2 and a mutation occurred at the polynucleotide sequence excluding the SH2 domain within the SOCS2;

(3) transducing the first expression vector in step (1) and each the second expression vector in step (2), together or one after another, into NK cells;

(4) measuring the amount of expression of Pyk2 protein in transduced cells (experimental group) in step (3); and

(5) selecting a mutant SOCS2 having the decreased amount of expression of Pyk2 protein compared to control.

Yet another object of the present invention is to provide a method for screening SOCS2 having the increased NK cell-activating effect, comprising the steps of:

(1) preparing a first expression vector wherein the Pyk2 gene having polynucleotide sequence of SEQ ID NO:18 is operably linked;

(2) preparing second expression vectors wherein a polynucleotide is operably linked, the polynucleotide encoding a mutant SOCS2 in which a SH2 domain encoded by the polynucleotide of SEQ ID NO:21 is conserved and a mutation occurred at the polynucleotide sequence excluding the SH2 domain within SOCS2;

(3) transducing the first expression vector in step (1) and each the second expression vector in step (2), together or one after another, into NK cells; and

(4) determining whether the activity of transduced NK cells in step (3) increased or not compared to control.

Further another object of the present invention is to provide a pharmaceutical composition for preventing or treating cancer, comprising the NK cells as an active ingredient.

In order to achieve the objects, the present invention provides a pharmaceutical composition for activating NK cells, comprising an expression vector wherein the socs2 (suppressor of cytokine signaling 2) gene having polynucleotide sequence of SEQ ID NO:1 is operably linked or SOCS2 protein encoded by the socs2 gene as an active ingredient.

The present invention also provides a method for activating NK cells comprising treating NK cells in vitro with SOCS2 protein comprising an SH2 (Src homology 2) domain encoded by a nucleic acid molecule having polynucleotide sequence of SEQ ID NO:21, and NK cells activated by the method.

Furthermore, the present invention provides a method for activating NK cells comprising the steps of:

(1) preparing an expression vector wherein the socs2 gene having polynucleotide sequence of SEQ ID NO:1 is operably linked; and

(2) transducing the expression vector prepared in step (1) into NK cells,

and NK cells activated by the method.

The present invention also provides a method for screening a mutant SOCS2 having the increased NK cell-activating effect, comprising the steps of:

(1) preparing a first expression vector wherein the Pyk2 gene having polynucleotide sequence of SEQ ID NO:18 is operably linked;

(2) preparing second expression vectors wherein a polynucleotide is operably linked, the polynucleotide encoding a mutant SOCS2 in which a SH2 domain encoded by the polynucleotide of SEQ ID NO:21 is conserved within SOCS2 and a mutation occurred at the polynucleotide sequence excluding the SH2 domain within the SOCS2;

(3) transducing the first expression vector in step (1) and each the second expression vector in step (2), together or one after another, into NK cells;

(4) measuring the amount of expression of Pyk2 protein in transduced cells (experimental group) in step (3); and

(5) selecting a mutant SOCS2 having the decreased amount of expression of Pyk2 protein compared to control.

Furthermore, the present invention provides a method for screening SOCS2 having the increased NK cell-activating effect, comprising the steps of:

(1) preparing a first expression vector wherein the Pyk2 gene having polynucleotide sequence of SEQ ID NO:18 is operably linked;

(2) preparing second expression vectors wherein a polynucleotide is operably linked, the polynucleotide encoding a mutant SOCS2 in which a SH2 domain encoded by the polynucleotide of SEQ ID NO:21 is conserved and a mutation occurred at the polynucleotide sequence excluding the SH2 domain within SOCS2;

(3) transducing the first expression vector in step (1) and each the second expression vector in step (2), together or one after another, into NK cells; and

(4) determining whether the activity of transduced NK cells in step (3) increased or not compared to control.

The present invention also provides a pharmaceutical composition for preventing or treating cancer, comprising the NK cells as an active ingredient.

The inventors of the present invention observed that when NK cells were treated with IL-15, a cytokine involved in NK cell differentiation, the expression of SOCS2 increased and the expression of proline-rich tyrosine kinase 2 (Pyk2) was inhibited by the SOCS2, the expression of which increased. In addition, when Pyk2 was overexpressed, the ability to produce IFN-γ and the ability to kill tumor cells of NK cells decreased. Therefore, SOCS2 can be used for activating NK cells and the NK cells activated by the method can be used for the prevention or treatment of cancer.

### BRIEF DESCRIPTION OF THE DRAWINGS

The above and other objects, features and other advantages of the present invention will be more clearly understood from the following detailed description taken in conjunction with the accompanying drawings, in which:

FIG. 1 shows (a) the result of measuring the mRNA expression of SOCS2 and (b) the result of examining SOCS2 protein expression during in vitro differentiation of NK cells for the examination of SOCS2 gene expression aspect in NK cells.

FIG. 2 shows (a) the result of measuring the mRNA expression of SOCS2 by IL-15 treatment and (b) the result that when NK-92 cells were treated with IL-7, IL-12, IL-15, IL-18, or IL-21, the mRNA expression of SOCS2 increased specifically by IL-15.

FIG. 3 shows (a) the result that the mRNA expression of SOCS2 by IL-15 increased specifically compared to those of SOCS1 or SOCS3 and (b) the result that when NK-92 cells and primary NK cells were treated with IL-15, the protein expression of SOCS2 increased.

FIG. 4 shows the result of FACS analysis for the effect of inhibition of SOCS2 expression on in vitro NK cell differentiation induced by IL-15.

FIG. 5 shows the result of measuring the phosphorylation of STAT5 using Western blot analysis. In order to investigate of the effect of inhibition of SOCS2 expression on IL-15 receptor signal transduction, SOCS2 expression was inhibited in NK cells and then, NK cells were treated with IL-15.

FIG. 6 shows the result of FACS analysis for the effect of inhibition of SOCS2 expression on IL-15 dependent NK cell survival.

FIG. 7 shows (a) the result of measuring the effect of inhibition of SOCS2 expression on IL-15-dependent NK cell differentiation and (b) the result of FACS analysis for the effect of inhibition of SOCS2 expression on the expression of various receptors of NK cells.

FIG. 8 shows (a) the result of measuring the effect of inhibition of SOCS2 expression on the ability to kill tumor cells (cytotoxicity) of NK-92 cells and (b) the result of measuring the effect of inhibition of SOCS2 expression on the cytotoxicity of mature NK cells.

FIG. 9 shows (a) the result of measuring the concentration of IFN-γ using ELISA and (b) the result of real time PCR analysis for the effect on mRNA expression of IFN-γ in order to investigate the effect of inhibition of SOCS2 expression on IFN-γ production in NK-92 cells.

FIG. 10 shows the result of measuring the effect of inhibition of SOCS2 expression on IFN-γ production in mature NK cells using ELISA.

FIG. 11 shows the result of Western blot analysis for the effect of inhibition of SOCS2 expression on NK-92 cell activating signal transduction mediated by various ligands on the surface of K562 cells.

FIG. 12 shows the result of Western blot analysis for the effect of inhibition of SOCS2 expression on NK92 cell activating signal transduction induced by NKp30 receptor stimulation.

FIG. 13 shows (a) the cytotoxicity and (b) IFN-γ production of NK-92 cells. NK-92 cells were treated with inhibitors of MAPKs (ERK, JNK, and p38) which were reported to play important roles in NK cell activating signal transduction.

FIG. 14 shows the result of examining the binding between Pyk2 protein and SOCS2 protein using a yeast two-hybrid screening.

FIG. 15 shows (a) the result of a GST pull down assay for examining the binding between SOCS2 and Pyk2 and (b) the result of performing immunoprecipitation with anti-Flag antibody. In order to observe the binding between SOCS2 and Pyk2 in cells, GST-SOCS2 and Flag-Pyk2 were overexpressed in 293T cells prior to the GST pull down assay and the immunoprecipitation.

FIG. 16 show (a) the result of a GST pull down assay for determining which domain of SOCS2 binds to Pyk2 and (b) the result of examining the endogenous binding between SOCS2 and Pyk2 in NK cells using immunoprecipitation. SOCS2 deletion mutants (GST-SOCS2-SOCS2, GST-SOCS2-SH2) and Flag-Pyk2 were overexpressed in 293T cells prior to the GST pull down assay and the immunoprecipitation.

FIG. 17 shows the result of identifying a binding motif of Pyk2 to SOCS2 by overexpressing Flag-Pyk2, Flag-Pyk2-Y402F and GST-SOCS2.

FIG. 18(a) shows the result of the hourly observation for the level of SOCS2 protein and p-Pyk2 protein using Western blot analysis. In order to observe the regulation of Pyk2 by SOCS2 in NK cells, NK-92 cells were treated with IL-15. FIG. 18(b) shows the result of the hourly observation for the level of SOCS2 and phosphorylated Pyk2 (p-Pyk2^{Tyr402}) in primary NK cells using Western blot analysis.

FIG. 19 shows the result of the observation for ubiquitination of Pyk2 after IL-15 treatment in NK-92 cells using immunoprecipitation analysis.

FIG. 20 shows the result of the observation for the level of Pyk2 protein when the SOCS2 expression was inhibited in NK cells using Western blot analysis.

FIG. 21 shows the result of the verification of the overexpression of Pyk2 using Western blot analysis. In order to verify whether overexpressed Pyk2 affected the activity of NK cells, GFP-Pyk2 was overexpressed in NK-92 cells.

FIG. 22 shows (a) the result of examining the effect of Pyk2 overexpression on the cytotoxicity of NK-92 cells using ⁵¹Cr-release analysis and (b) the result of measuring the effect of Pyk2 overexpression on IFN-γ production of NK-92 cells using ELISA.

### DESCRIPTION OF THE PREFERRED EMBODIMENTS

Features and advantages of the present invention will be more clearly understood by the following detailed description of the present preferred embodiments by reference to the accompanying drawings. It is first noted that terms or words used herein should be construed as meanings or concepts corresponding with the technical sprit of the present invention, based on the principle that the inventor can appropriately define the concepts of the terms to best describe his own invention. Also, it should be understood that detailed descriptions of well-known functions and structures related to the present invention will be omitted so as not to unnecessarily obscure the important point of the present invention.

Hereinafter, the present invention will be described in detail.

The present invention provides a pharmaceutical composition for activating natural killer (NK) cells, comprising an expression vector wherein the socs2 (suppressor of cytokine signaling 2) gene having polynucleotide sequence of SEQ ID NO:1 is operably linked or SOCS2 protein encoded by the socs2 gene as an active ingredient.

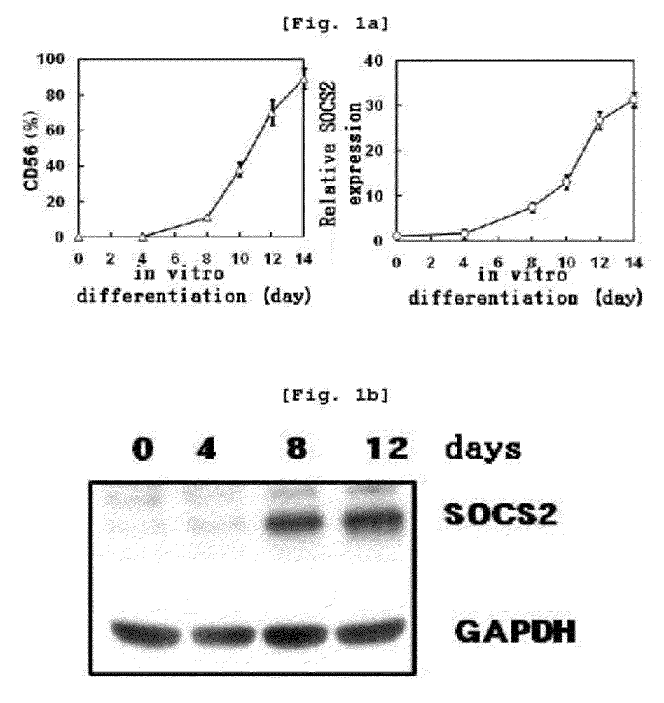

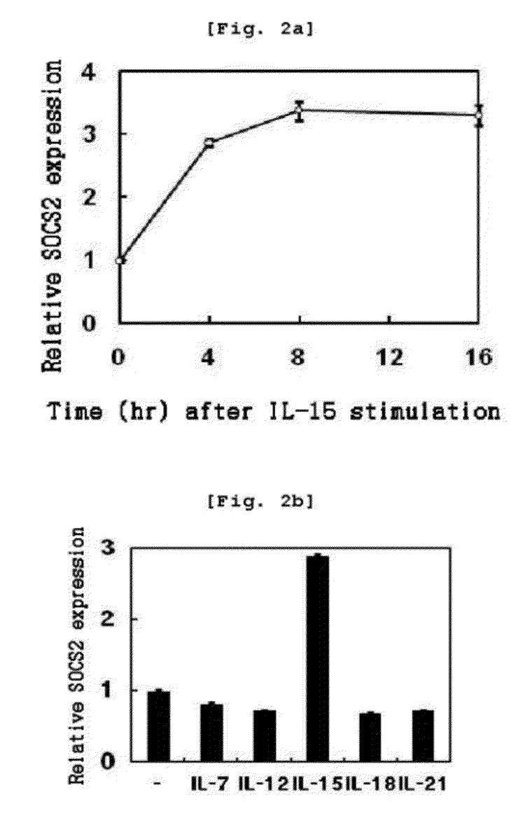

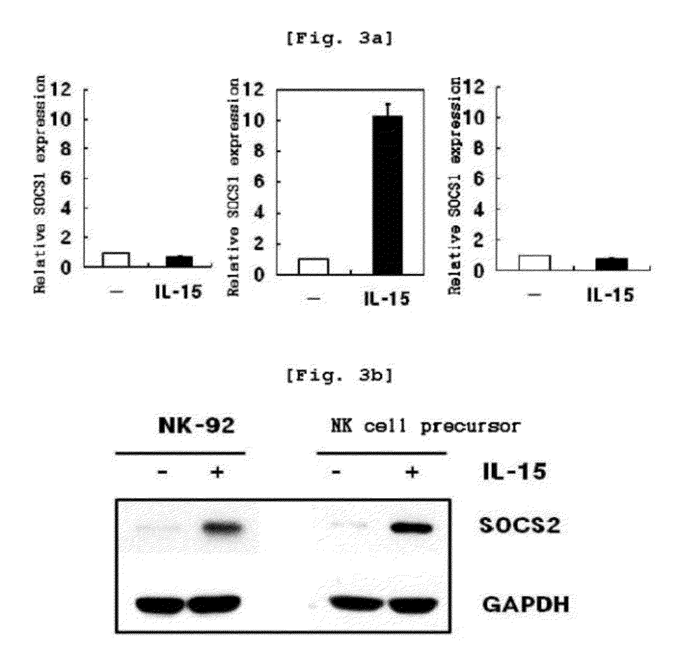

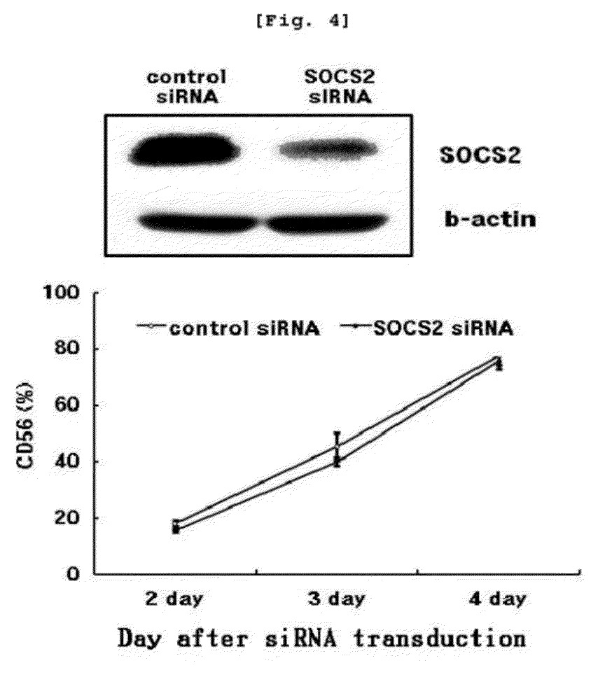

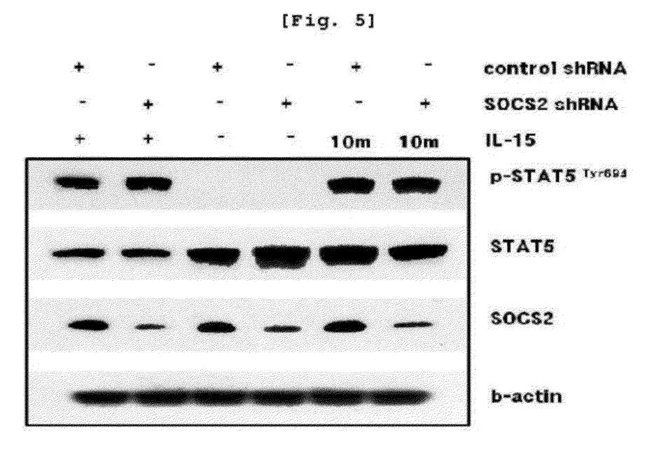

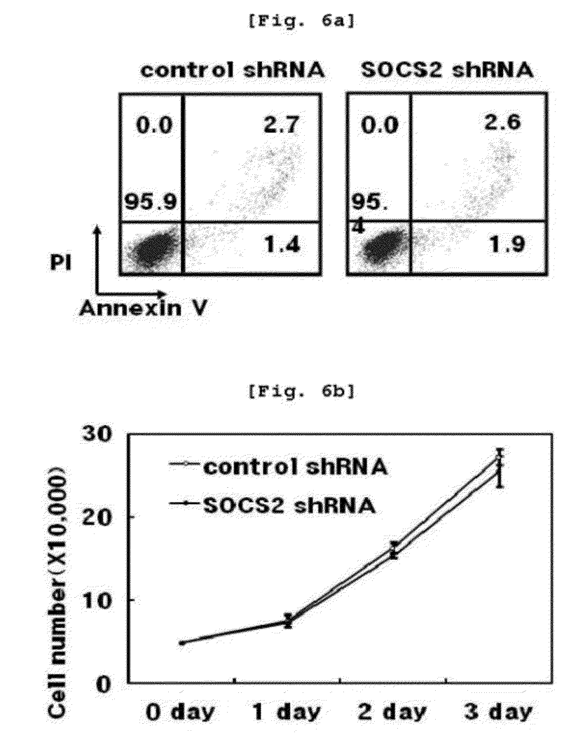

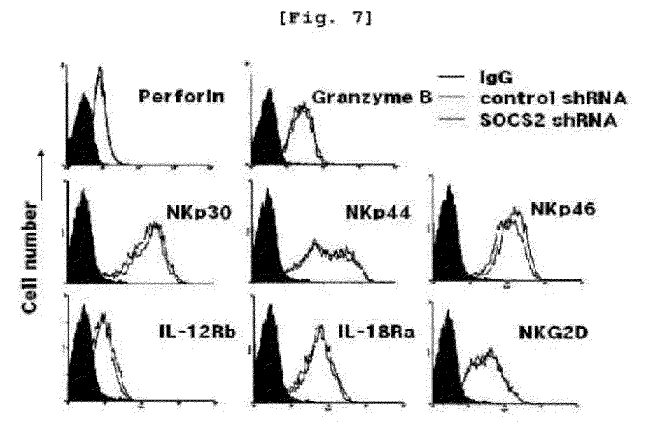

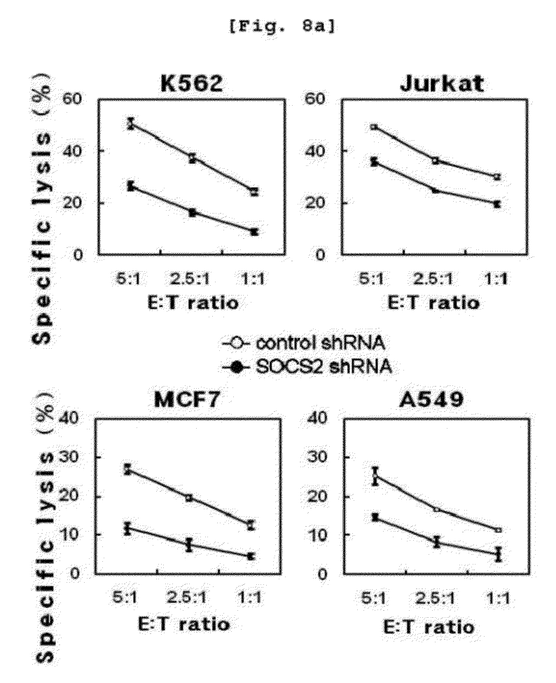

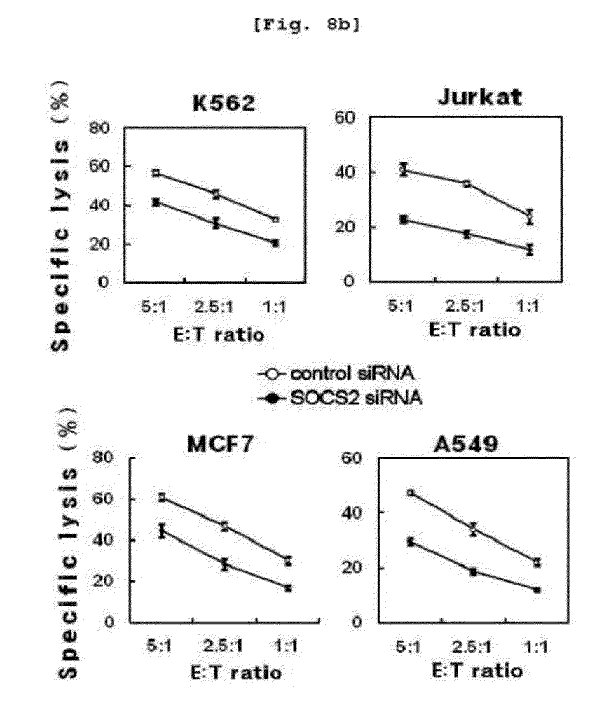

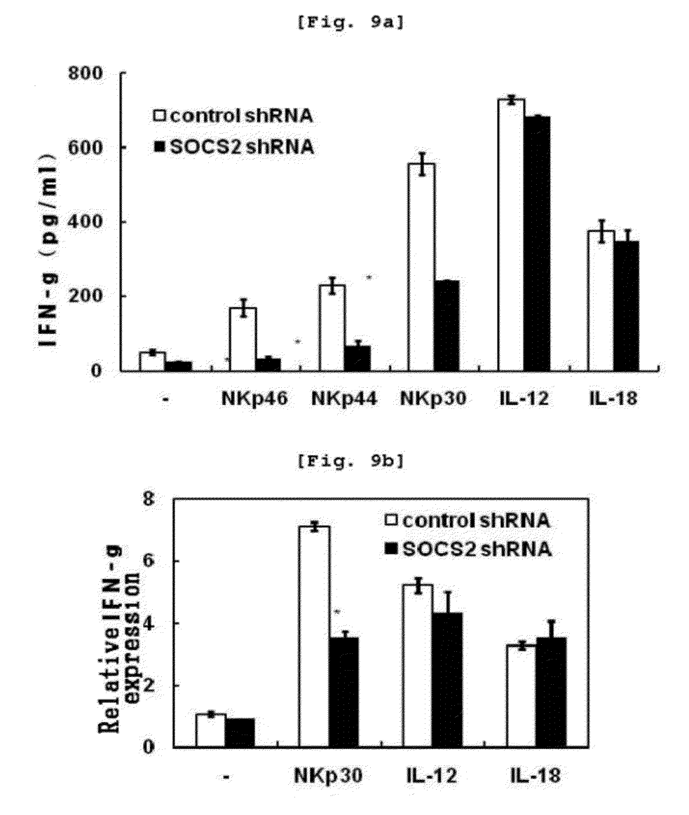

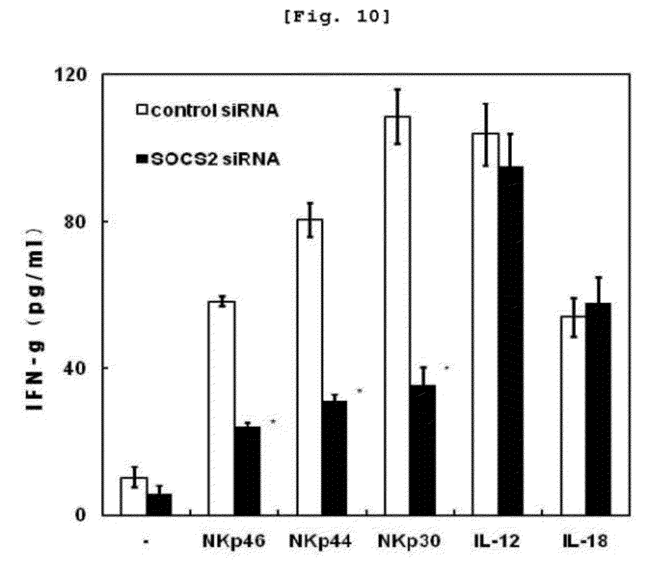

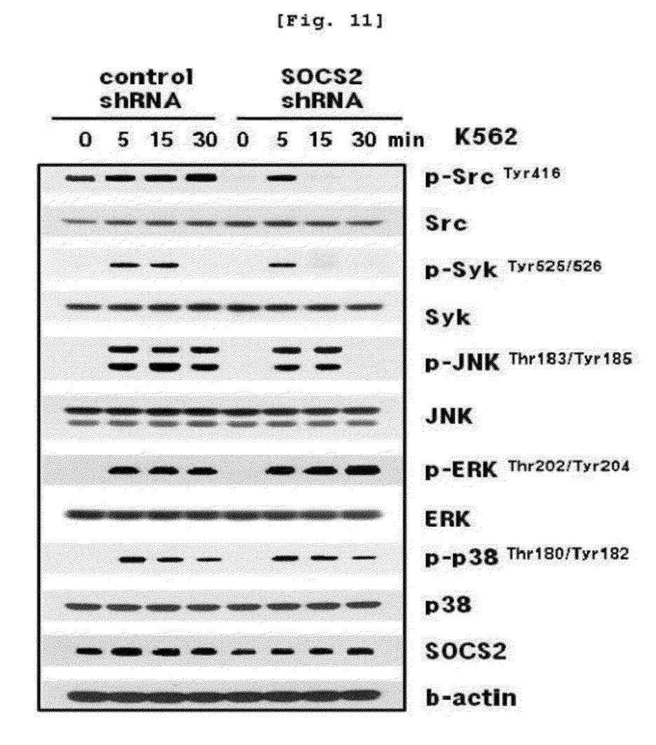

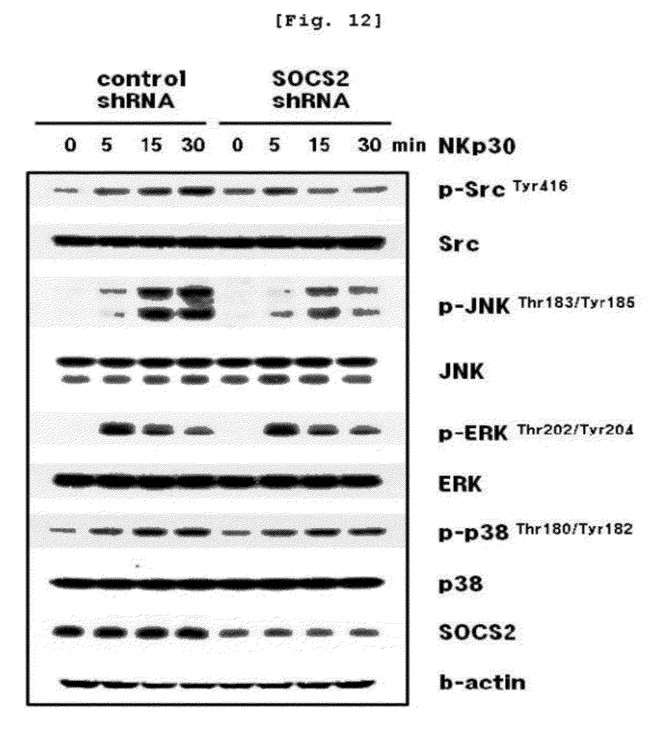

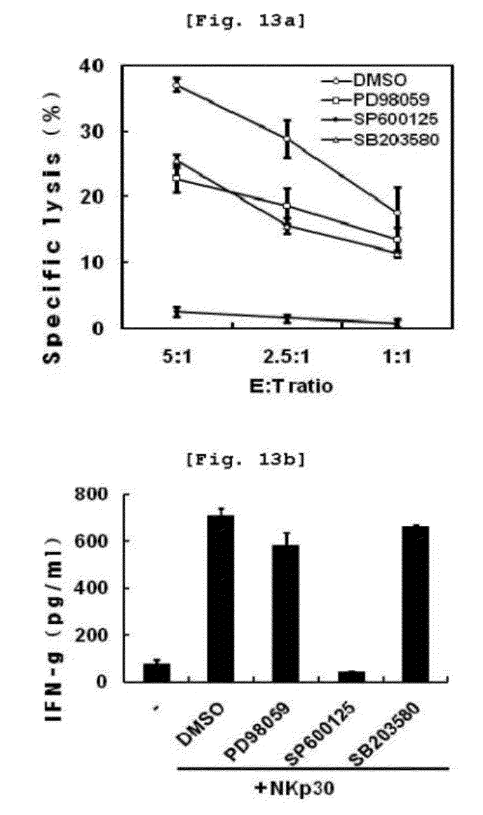

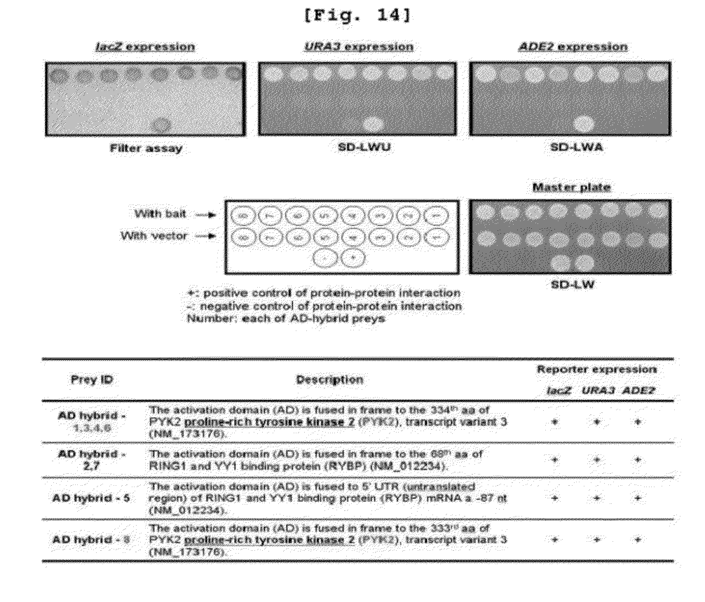

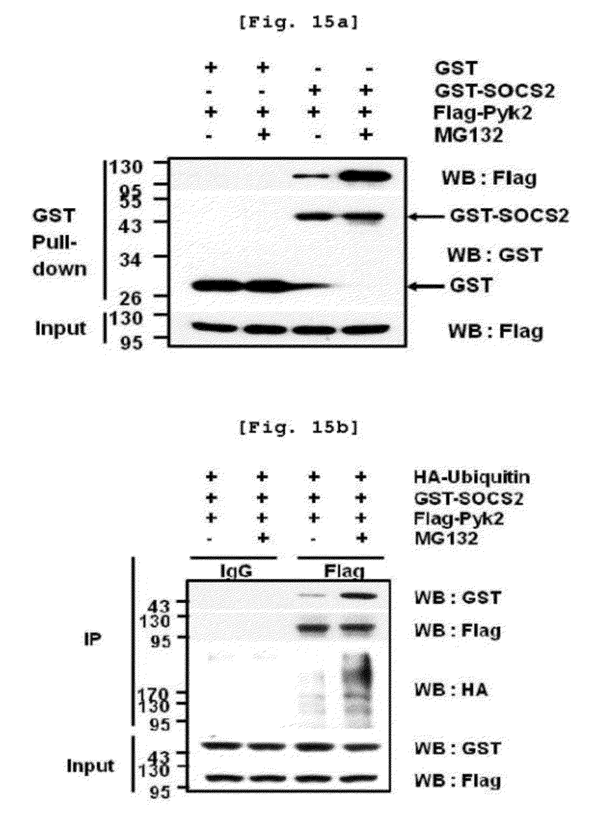

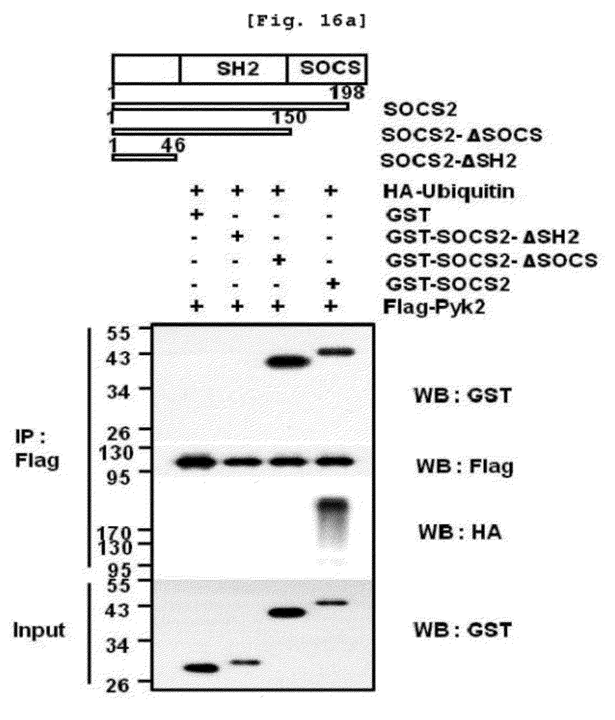

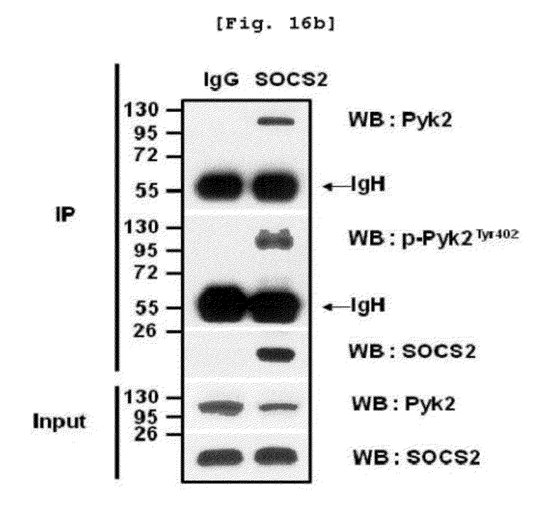

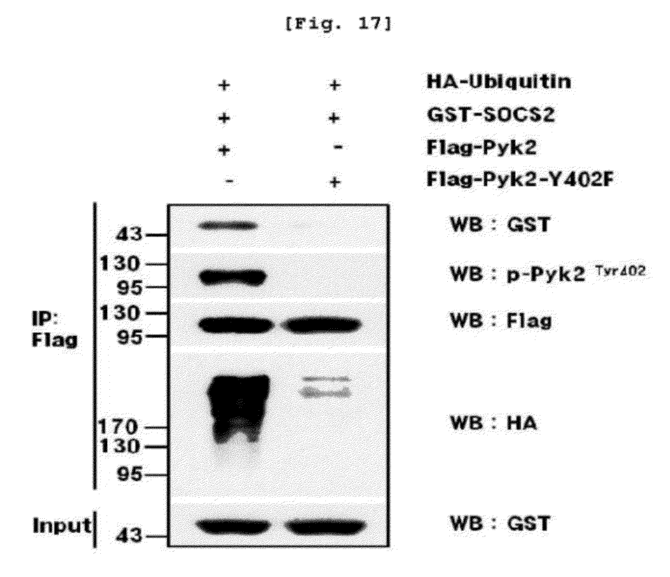

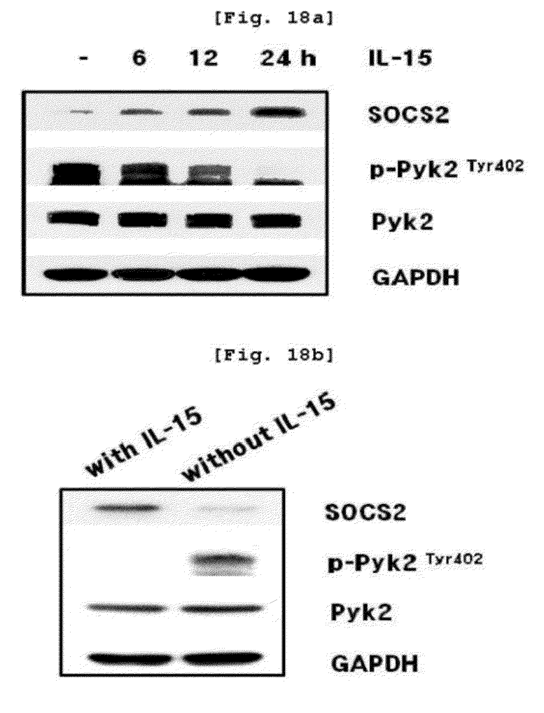

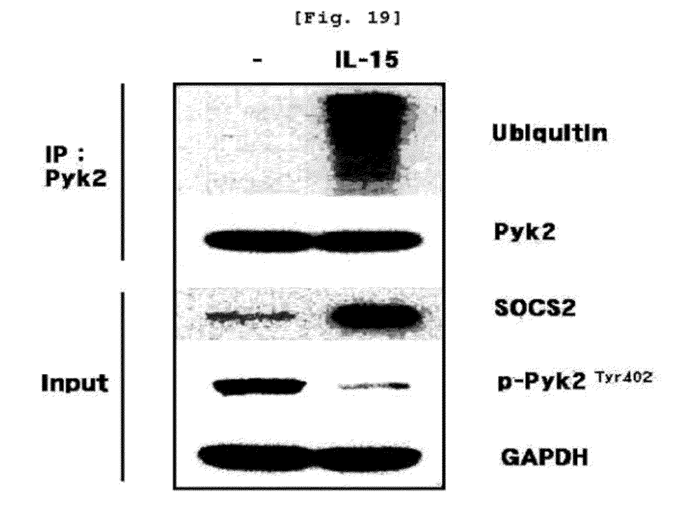

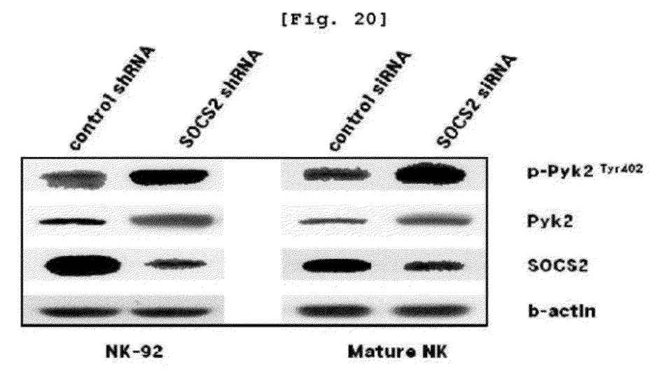

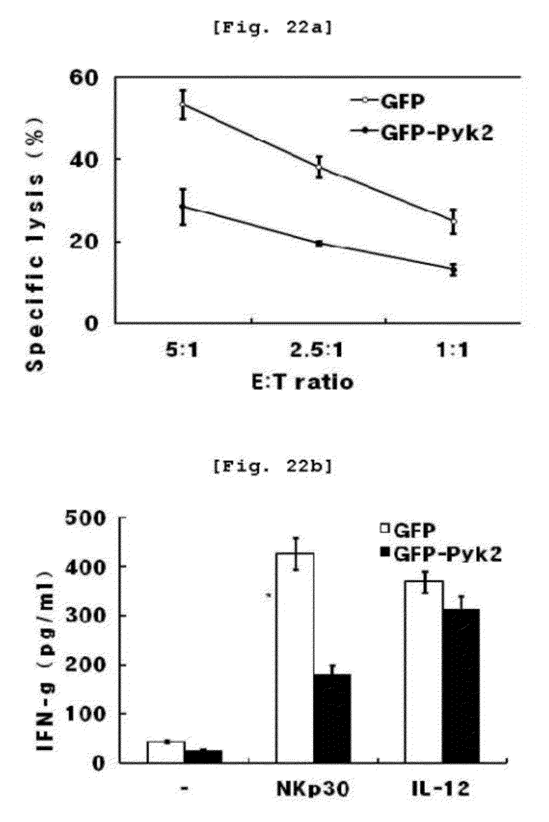

In specific embodiments of the present invention, the present inventors found that SOCS2 (suppressor of cytokine signaling 2) expression increased following NK cell differentiation (FIG. 1(a) and FIG. 1(b)); SOCS2 expression was induced by IL-15 (Interleukin-15), the cytokine priming NK cell differentiation (FIG. 2(a) and FIG. 2(b)); SOCS2 expression was regulated mutually and specifically between IL-15 and SOCS2 (FIG. 3(a) and FIG. 3(b)). In addition, the present inventors found that when SOCS2 expression was inhibited, differentiation (FIG. 4), receptor signal transduction (FIG. 5), proliferation (FIG. 6), and survival (FIG. 7) of NK cells were not affected, but NK cell cytotoxicity (FIG. 8(a) and FIG. 8(b)) and IFN-γ (Interferon-γ) production by NCR (natural cytotoxicity receptor) stimulation decreased (FIG. 9(a) and FIG. 9(b)), and the reduction in the IFN-γ production was from reduction in mRNA expression of IFN-γ (FIG. 10). In addition, the present inventors examined whether the reduction in NK cell activity induced by the inhibition of SOCS2 expression affected the intracellular signaling pathways or not and found that when SOCS2 expression was inhibited in NK cells, phosphorylation of Src (sarcoma, proto-oncogenic tyrosine kinases), Syk (Spleen tyrosine kinase) and JNK (c-Jun N-terminal kinases) was reduced (FIG. 11, FIG. 12, FIG. 13(a) and FIG. 13(b)). The binding of SOCS2 protein with Pyk2 (proline-rich tyrosine kinase 2) was reported through a yeast two-hybrid screening. The present inventors identified that SOCS2 and Pyk2 combine together in human cell line (293T) and NK cells (FIG. 15(a), FIG. 15(b), and FIG. 16(b)). In addition, the present inventors found that SOCS2 SH2 (Src homology 2) domain (FIG. 16(a)) and phosphorylation of Pyk2 (FIG. 17) are important in the binding between SOCS2 and Pyk2. The present inventors also found that when SOCS2 expression was inhibited in NK cells, the expression of Pyk2 and phosphorylated Pyk increased (FIG. 18(a) and FIG. 18(b)). That is because SOCS2 induces the ubiquitin-mediated proteasomal degradation of Pyk2 (FIG. 19 and FIG. 20). The present inventors found that the reduction in NK cell activity by the inhibition of SOCS2 expression was also because SOCS2-mediated Pyk2 regulation was broken (FIG. 22(a) and FIG. 22(b)).

Therefore, since SOCS2 expression increases during IL-15-induced NK cell priming and the increased SOCS2 regulates phosphorylated Pyk2 to maintain NK cell activity, SOCS2 can be used for a pharmaceutical composition for activating NK cells.

The pharmaceutical composition for activating NK cells of the present invention may be treated in vitro, in vivo, or ex vivo. Examples of methods of treating the pharmaceutical composition of the present invention include, but are not limited to, treating the pharmaceutical composition of the present invention in vitro to activate NK cells and administering the NK cells to an individual; administering the pharmaceutical composition directly to an individual to activate NK cells (in vivo); and collecting NK cells from an individual, treating NK cells with the pharmaceutical composition of the present invention to activate, and then putting NK cells back into the individual (ex vivo). The methods of treating the pharmaceutical composition may be selected by those skilled in the art depending on diseases, ages, gender, and body weight of the individual, etc. The individual may be mammals. Examples of typical mammals include, but are not limited to, humans, nonhuman primates, mice, rats, dogs, cats, horses, and cattle. The diseases may be, but are not limited to, various diseases related with tumor, for example, various solid cancers including lung cancer, liver cancer, stomach cancer, colon cancer, bladder cancer, prostate cancer, breast cancer, ovarian cancer, cervical cancer, thyroid cancer, melanoma, etc. as well as various hematologic malignancy including leukemia, and preferably lung cancer, breast cancer, and hematologic malignancy. The pharmaceutical composition of the present invention may be administered orally or parenterally. For parenteral administration, topical application, or intra-abdominal injection, intra-rectal injection, subcutaneous injection, intravenous injection, intramuscular injection, or intrathoracic injection may be preferable.

The pharmaceutical composition may further comprise diluents, disintegrators, sweeteners, lubricants, aromatics, etc. that are conventionally used. Examples of disintegrators include sodium starch glycolate, Crospovidone, crosscarmellose sodium, alginic acid, carboxymethylcellulose calcium, carboxymethylcellulose sodium, chitosan, guar galactomannan, low substituted hydroxypropyl cellulose, aluminum magnesium silicate, polacrilin potassium, etc. In addition, the pharmaceutical composition may further comprise a pharmaceutically acceptable additive. Examples of pharmaceutically acceptable additives include starch, gelatinized starch, microcrystalline cellulose, lactose, povidone, colloidal silicon dioxide, dibasic calcium phosphate, mannitol, maltose, gum Arabic, starch pregelatinized, corn starch, cellulose powder, hydroxypropyl cellulose, Opadry, sodium starch glycolate, carnauba wax, aluminum silicate, stearic acid, magnesium stearate, aluminum stearate, calcium stearate, sucrose, glucose, sorbitol, talc, etc. The pharmaceutically acceptable additive according to the present invention may be included in the pharmaceutical composition in an amount of from about 0.1 to about 90 parts by weight with respect to the pharmaceutical composition.

Solid formulations for oral administration include powders, granules, tablets, capsules, soft capsules, pills, etc. Liquid formulation for oral administrations include suspensions, liquid for internal use, emulsions, syrups, aerosols, etc. and various excipients such as wetting agents, sweeteners, aromatics, preservatives, etc. in addition to generally-used simple diluents such as water and liquid paraffin may be included. Preparations for parenteral administration may be formulated as powders, granules, tablets, capsules, sterile solutions, liquid, water-insoluble excipients, suspensions, emulsions, syrups, suppositories, external preparations such as aerosols, etc., and sterile injections by general methods and preferably, skin external pharmaceutical compositions such as creams, gels, patches, sprays, ointments, plasters, lotions, liniments, pastes, and cataplasma may be prepared to use, but not limited to such. Propylene glycol, polyethylene glycol, vegetable oil such as olive oil, and injectable ester such as ethylolate, etc. may be used for water insoluble excipients and suspensions. Witepsol, macrogol, tween 61, cacao butter, laurin butter, glycerogelatin, etc. may be used for a suppository base.

The preferred administration dose of the pharmaceutical composition may be different depending on degrees of absorption of active ingredients in living bodies, inactivation ratio and excretion rate, age, gender, condition of the individual, and severity of disease to be treated, and be selected appropriately by those skilled in the art. For preferable effects for preparation for oral administration, the pharmaceutical composition may be administered generally for adults in a dose of from about 0.0001 to about 100 mg/kg body weight per day, preferably from about 0.001 to about 100 mg/kg. The administration frequency may be once a day or a few times a day. The administration dose is not intended to limit the scope of the present invention in any way.

The present invention also provides a method for activating NK cells comprising treating NK cells in vitro with SOCS2 protein comprising an SH2 (Src homology 2) domain encoded by a nucleic acid molecule having polynucleotide sequence of SEQ ID NO:21, and NK cells activated by the method.

NK cells can be activated with treatment of SOCS2 protein of SEQ ID NO:1, preferably, a mutant SOCS2 protein of SEQ ID NO:25. Any mutant SOCS2 protein may be used, provided that it comprises an SH2 domain which is important for the binding between SOCS2 and Pyk2.

The method for activating NK cells may further comprise examining whether the NK cells were activated or not after the above steps. Whether the NK cells were activated or not may be determined by, but is not limited to,

i) examining whether the expression of Pyk2 protein decreased or not;

ii) examining whether IFN-γ production of experimental group increased or not compared to control when NCR (natural cytotoxicity receptor) stimulation was given to cells; or

iii) examining whether the ability to kill target cells of experimental group increased or not compared to control. Those skilled in the art would know easily how to measure whether NK cells are activated or not.

Furthermore, the present invention provides a method for activating NK cells comprising the steps of:

(1) preparing an expression vector wherein the socs2 gene having polynucleotide sequence of SEQ ID NO:1 is operably linked; and

(2) transducing the expression vector prepared in step (1) into NK cells,

and NK cells activated by the method.

The present invention also provides a method for screening a mutant SOCS2 having the increased NK cell-activating effect, comprising the steps of:

(1) preparing a first expression vector wherein the Pyk2 gene having polynucleotide sequence of SEQ ID NO:18 is operably linked;

(2) preparing second expression vectors wherein a polynucleotide is operably linked, the polynucleotide encoding a mutant SOCS2 in which a SH2 domain encoded by the polynucleotide of SEQ ID NO:21 is conserved within SOCS2 and a mutation occurred at the polynucleotide sequence excluding the SH2 domain within the SOCS2;

(3) transducing the first expression vector in step (1) and each the second expression vector in step (2), together or one after another, into NK cells;

(4) measuring the amount of expression of Pyk2 protein in transduced cells (experimental group) in step (3); and

(5) selecting a mutant SOCS2 having the decreased amount of expression of Pyk2 protein compared to control.

SOCS2 in the step (2) may be a protein encoded by the polynucleotide of SEQ ID NO:1. The mutant SOCS2 in the step (2) may be a protein of which one or more amino acid residues are substituted to, added to, or deleted from, the SOCS2 encoded by the polynucleotide of SEQ ID NO:1, preferably, a protein encoded by the polynucleotide of SEQ ID NO:25.

In the step (4), whether protein expression of Pyk2 decreased or not may be examined by performing, but not limited to, any one method selected from the group consisting of Western blot analysis, immunostaining, fluorescent staining, and reporter assay. Any methods that are well-known in the art may be used.

The step of examining whether the NK cell activity increased actually or not when a test compound selected by the method for screening was treated may be further comprised. Whether the NK cell activity increased or not may be examined by, but is not limited to,

i) examining whether the expression of Pyk2 protein decreased or not;

ii) examining whether IFN-γ production of experimental group increased or not compared to control when NCR stimulation was given to cells; or

iii) examining whether the ability to kill target cells of experimental group increased or not compared to control. Those skilled in the art would know easily how to measure the ability of NK cells to kill target cells.

Furthermore, the present invention provides a method for screening SOCS2 having the increased NK cell-activating effect, comprising the steps of:

(1) preparing a first expression vector wherein the polynucleotide of SEQ ID NO:18 encoding Pyk2 gene is operably linked;

(2) preparing second expression vectors wherein a polynucleotide is operably linked, the polynucleotide encoding a mutant SOCS2 in which a SH2 domain encoded by the polynucleotide of SEQ ID NO:21 is conserved and a mutation occurred at the polynucleotide sequence excluding the SH2 domain within SOCS2;

(3) transducing the first expression vector in step (1) and each the second expression vector in step (2), together or one after another, into NK cells; and

(4) determining whether the activity of transduced NK cells in step (3) increased or not compared to control.

SOCS2 in the step (2) may be a protein encoded by the polynucleotide of SEQ ID NO:1. The mutant SOCS2 in the step (2) may be a protein of which one or more amino acid residues are substituted to, added to, or deleted from, the SOCS2 encoded by the polynucleotide of SEQ ID NO:1, preferably, a protein encoded by the polynucleotide of SEQ ID NO:25.

The present invention also provides a pharmaceutical composition for preventing or treating cancer, comprising the NK cells as an active ingredient.

The pharmaceutical composition of the present invention may be used for treating various diseases related with tumor, for example, various solid cancers including lung cancer, liver cancer, stomach cancer, colon cancer, bladder cancer, prostate cancer, breast cancer, ovarian cancer, cervical cancer, thyroid cancer, melanoma, etc. as well as various hematologic malignancy including leukemia, and preferably lung cancer, breast cancer, and hematologic malignancy. The pharmaceutical composition of the present invention may be administered orally or parenterally. For parenteral administration, topical application, or intra-abdominal injection, intra-rectal injection, subcutaneous injection, intravenous injection, intramuscular injection, or intrathoracic injection may be preferable.

The pharmaceutical composition of the present invention may be administered to mammals. Examples of typical mammals include, but are not limited to, humans, nonhuman primates, mice, rats, dogs, cats, horses, and cattle. The pharmaceutical composition of the present invention may be administered orally or parenterally. For parenteral administration, topical application, or intra-abdominal injection, intra-rectal injection, subcutaneous injection, intravenous injection, intramuscular injection, or intrathoracic injection may be preferable.

The pharmaceutical composition may further comprise diluents, disintegrators, sweeteners, lubricants, aromatics, etc. that are conventionally used. Examples of disintegrators include sodium starch glycolate, Crospovidone, crosscarmellose sodium, alginic acid, carboxymethylcellulose calcium, carboxymethylcellulose sodium, chitosan, guar galactomannan, low substituted hydroxypropyl cellulose, aluminum magnesium silicate, polacrilin potassium, etc. In addition, the pharmaceutical composition may further comprise a pharmaceutically acceptable additive. Examples of pharmaceutically acceptable additives include starch, gelatinized starch, microcrystalline cellulose, lactose, povidone, colloidal silicon dioxide, dibasic calcium phosphate, mannitol, maltose, gum Arabic, starch pregelatinized, corn starch, cellulose powder, hydroxypropyl cellulose, Opadry, sodium starch glycolate, carnauba wax, aluminum silicate, stearic acid, magnesium stearate, aluminum stearate, calcium stearate, sucrose, glucose, sorbitol, talc, etc. The pharmaceutically acceptable additive according to the present invention may be included in the pharmaceutical composition in an amount of from about 0.1 to about 90 parts by weight with respect to the pharmaceutical composition.

Solid formulations for oral administration include powders, granules, tablets, capsules, soft capsules, pills, etc. Liquid formulation for oral administrations include suspensions, liquid for internal use, emulsions, syrups, aerosols, etc. and various excipients such as wetting agents, sweeteners, aromatics, preservatives, etc. in addition to generally-used simple diluents such as water and liquid paraffin may be included. Preparations for parenteral administration may be formulated as powders, granules, tablets, capsules, sterile solutions, liquid, water-insoluble excipients, suspensions, emulsions, syrups, suppositories, external preparations such as aerosols, etc., and sterile injections by general methods and preferably, skin external pharmaceutical compositions such as creams, gels, patches, sprays, ointments, plasters, lotions, liniments, pastes, and cataplasma may be prepared to use, but not limited to such. Propylene glycol, polyethylene glycol, vegetable oil such as olive oil, and injectable ester such as ethylolate, etc. may be used for water insoluble excipients and suspensions. Witepsol, macrogol, tween 61, cacao butter, laurin butter, glycerogelatin, etc. may be used for a suppository base.

The preferred administration dose of the pharmaceutical composition may be different depending on degrees of absorption of active ingredients in living bodies, inactivation ratio and excretion rate, age, gender, condition of the individual, and severity of disease to be treated, and be selected appropriately by those skilled in the art. For preferable effects for preparation for oral administration, the pharmaceutical composition may be administered generally for adults in a dose of from about 0.0001 to about 100 mg/kg body weight per day, preferably from about 0.001 to about 100 mg/kg. The administration frequency may be once a day or a few times a day. The administration dose is not intended to limit the scope of the present invention in any way.

### EXAMPLES

Hereinafter, the present invention will be described in more detail with reference to the following examples.

However, the following examples are provided for illustrative purposes only, and the scope of the present invention should not be limited thereto.

### Example 1

### Cell Culture

<1-1> Cell Line Culture

Cell lines of Table 1 were purchased from American Type Culture Collection (ATCC) and cultured at 37° C., 5% CO₂.

The cultured cell lines were detached from 75-cell culture flask with Trypsin-EDTA (Trypsin-ethylenediamine tetraacetic acid, Invitrogen, U.S.A.) treatment and serum-containing medium was added to inactivate trypsin. Cells were centrifuged to precipitate. After removing supernatant, culture medium depending on each cell line was added to suspend cells. Live cells were counted via the trypan blue dye exclusion method using a hemocytometer. Then cells were subcultured in 100 mm dishes at 5×10⁵ cells/flask.

TABLE 1

| | | | Culture |
|---|---|---|---|
| Cell line | Cell type | ATCC No. | medium |
| K562 | chronic myelogenous leukemia | CCL-243 ™ | IMDM |
| Jurkat | acute T cell leukemia | TIB-152 ™ | RPMI- |
| | | | 1640 |
| MCF7 | breast adenocarcinoma | HTB-22 ™ | EMEM |
| A549 | lung carcinoma | CCL-185 ™ | F-12K |
| NK-92 | malignant non-Hodgkin's | CRL-2407 ™ | AMEM |
| | lymphoma (NK cell) | | |
| HEK293T | kidney epithelial | CRL-11268 ™ | DMEM |

IMDM(Iscove's Modified Dulbecco's Medium, Gibco, U.S.A.): IMDM containing 10% FBS(Gibco)
RPMI-1640: RPMI-1640 containing 10% FBS
EMEM(Eagle's Minimum Essential Medium, Gibco): EMEM containing 0.01 mg/ml bovine insulin and 10% FBS
F-12K: F-12K containing 10% FBS
AMEM(Alpha Minimum Essential medium, Gibco): AMEM containing 2 mM L-glutamine(Gibco), 1.5 g/L sodium bicarbonate(Gibco), 0.2 mM inositol(Gibco), 0.1 mM 2-mercaptoethanol(Gibco), 0.02 mM folic acid(Gibco), 100-200 U/ml recombinant IL-2(Gibco), 12.5% horse serum(Gibco) and 12.5% FBS
DMEM(Dulbecco's Modified Eagle's Medium, Gibco): DMEM containing 10% FBS

<1-2> Primary Cell Culture

Primary NK cells and mature NK cells were harvested from mothers' umbilical cord blood. Cells were obtained from mothers' umbilical cord blood using Histopaque-1077 (Sigma, U.S.A) and then, the NK cells were isolated using the human NK Cell Isolation Kit (Miltenyi, Germany). The NK cells centrifuged and stored at −70° C. in a polypropylene container. Harvest of primary NK cells and mature NK cells was carried out after obtaining prior written consent. The local Institutional Review Board approved the collection of biochemical materials and information from these patients for research purposes. The harvested primary NK cells and mature NK cells were cultured at 37° C., 5% CO₂ and used for the following experimental.

### Example 2

### Preparation of SOCS2 shRNA-expressing Virus

The pLK0.1-SOCS2 shRNA vector (TRCN0000057058) which express shRNA (SEQ ID NO.2: 5′-CCGGCGCATTCAGACTACCTACTAACTCGAGTTAGTAGGTAGTCTGAATGCGTTTTTG-3′) for socs2(suppressor of cytokine signaling 2)(SEQ ID NO:1) and pLK0.1-nontarget shRNA control vector (SHC002) which express negative control shRNA (SEQ ID NO.3: 5′-CCGGCAACAAGATGAAGAGCACCAACTCGAGTTGGTGCTCTTCATCTTGTTGTTTTT-3′) were purchased from Sigma (U.S.A.). Lentiviruses which express SOCS2 shRNA or control shRNA were prepared using the vectors, third-generation packaging system (pMDLg/pRRE, pRSV-Rev, pMD2.G) and HEK293T cell line according to manufacturer's instructions. Lentivirus-containing HEK293T cell culture media was concentrated by ultracentrifugation at 50,000×g for 90 min at 4° C. Then, titer of the lentivirus concentrate was determined using Lenti-X™ p24 Rapid Titer Kit(clontech, U.S.A.)

TABLE 2

| Gene | Sense primer | | Antisense primer | |
|---|---|---|---|---|
| SOCS2 | SEQ ID NO: 4 | 5′-taaaagaggcaccagaaggaac-3′ | SEQ ID NO: 5 | 5′-tcgatcagatgaaccacactg-3′ |
| GAPDH | SEQ ID NO: 6 | 5′-cagcctcaagatcatcagca-3′ | SEQ ID NO: 7 | 5′-gtcttctgggtggcagtgat-3′ |

<2-1> Western Blot Analysis

Each NK cell was lysed in RIPA buffer (50 mM Tris-HCl, pH 7.4, 150 mM NaCl, 0.25% SDS, 1% NP-40, 1 mM EDTA, a protease inhibitor cocktail, and a phosphate inhibitor cocktail) and the concentration of protein present in the whole cell lysate was determined using BCA Protein Assay Kit (Pierce, U.S.A.). 30 μg of each protein sample was resolved using 10 or 12% SDS-PAGE gel and transferred to an Immobilon-P membrane (Millipore Corporation, U.S.A.). The membrane was blocked with 5% skim milk and treated with anti-SOCS2 antibody (Santa Cruz, U.S.A.) as a primary antibody for 1 day at 4° C. HRP-conjugated anti-rabbit secondary antibody (Santa Cruz, U.S.A.) was attached to the primary antibody-treated membrane, and then, Immobilon Western Chemiluminescent HRP Substrate (Milipore Corporation) was added thereto. The membrane was exposed to X-ray film. As quantitative control, the amount of expression of GAPDH was examined using anti-GAPDH primary antibody (Santa Cruz, U.S.A.) by the above method. The result was shown in FIG. 1(b).

Consequently, as shown in FIG. 1(b), SOCS2 protein which was hardly detected was detected in NK cells differentiated for 8 days or more. From this, it was found that as differentiation proceeded, mRNA and protein expression of socs2 increased, and especially from 8th day, they increased sharply.

<2-2> Determination of Induction of SOCS2 Expression by IL-15 in NK Cells

NK cell differentiation is mediated by IL-15 (Interleukin 15) cytokine. To determine whether the SOCS2 upregulation following NK cell differentiation was induced by IL-15, the present inventors experimented on the IL-15-induced SOCS2 upregulation in mature NK cells. The experimented NK-92 human NK cells and primary NK cells live and proliferate in the condition where IL-15 exists. Accordingly, to measure the effect of IL-15, deprivation of IL-15 was carried out by culturing cells in a medium without IL-15 for 24 hr prior to IL-15 treatment, and then the medium was replaced with a medium containing 10 ng/mL IL-15 to make a cell differentiation condition. Real time PCR was carried out with NK-92 cells at 4, 8, 12, and 16th hr after the replacement of the medium using the same method in Example <3-1-1> to measure the amount of socs2 mRNA expression. To determine whether SOCS2 expression is upregulated by other cytokines as well as IL-15, deprived NK-92 cells were treated with 30 ng/mL of IL7 (Peprotech), IL-12 (Peprotech), IL-15, IL-18 (Peprotech), and IL-21 (Peprotech) for 16 hr and real time PCR was carried out with NK-92 cells using the same method in Example <3-1-1> to measure the amount of SOCS2 expression.

Consequently, as shown in FIG. 2(a), it was found that by 4 hr after the IL-15 treatment, SOCS2 expression increased in NK-92 cells, and then, the amount of SOCS2 expression was maintained. Also, as shown in FIG. 2(b), it was found that the SOCS2 expression was induced specifically by IL-15 stimulation.

<2-3> Determination of Induction of SOCS Family Expression by IL-15 in NK Cells

To determine whether the expression of other SOCS family genes is also induced by IL-15, the present inventors carried out the following experiment. Deprivation and IL-15 stimulation was carried out with primary NK cells using the same method in Example <2-2>. The amounts of mRNA expression of socs1, socs2 and socs3 were measured by real time PCR. Primers in Table 1 and Table 3 were used.

Also, to determine whether the induction of SOCS2 expression by IL-15, which were confirmed in FIG. 2(a) and FIG. 2(b), can be observed in NK-92 cells as well as primary NK cells, Western blot analysis was carried out using the same method in Example <2-1> with NK-92 cells and primary NK cells which were deprived of IL-15 and then stimulated with IL-15 as the same method in Example <2-2> to observe SOCS2 protein expression.

Consequently, only socs2 mRNA expression among SOCS family members increased in primary NK cells by IL-15 stimulation, as shown in FIG. 3(a). As confirmed in FIG. 3(b), IL-15 stimulated SOCS2 expression was observed in both NK-92 cells and primary NK cells. Therefore, it was confirmed that IL-15 cytokine stimulation increases socs2 mRNA and protein expression, and socs2 mRNA and protein expressions are regulated mutually and specifically between IL-15 and SOCS2.

### Example 3

### Increase in socs2 Gene Expression by IL-15

<3-1> Determination of Increase in SOCS2 Gene Expression Following NK Cell Differentiation

To analyze socs2 gene expression aspect in NK cell differentiation, the present inventors cultured cells in the primary NK cell differentiation condition of Example <1>. Specifically, CD34+ hematopoietic stem cells were isolated from mothers' umbilical cord blood using a Human CD34 Isolation Kit. Isolated CD34+ hematopoietic stem cells were differentiated into NK cell precursors by incubating the cells in a medium supplemented with SCF (30 ng/mL, Peprotech, U.S.A.) and Flt3-ligand (50 ng/mL, Peprotech) for 14 days. NK cell precursors were differentiated into NK cells by incubating the NK cell precursors in a medium supplemented with IL-15 (30 ng/mL, Peprotech) for 14 days. Through real time PCR, socs2 mRNA expression was determined with NK cells at 0, 2, 4, 6, 8, 10, 12 and 14th day after differentiation. SOCS2 protein expression was determined with NK cells at 0, 4, 8, and 12th day by Western blot analysis. Through FACS analysis, the expression of NK cell surface marker CD56 was also determined with NK cells at 0, 2, 4, 6, 8, 10, 12 and 14th day after differentiation (FIG. 1(a)).

<3-1-1> Real Time PCR

Specifically, each NK cell was collected and total RNA was extracted using Trizol Reagent (Invitrogen, U.S.A.). Then, 1 μg of the RNA was reverse transcribed into cDNA using reverse transcriptase superscript II (Invitrogen). Using the cDNA as template, real time PCR was performed with the 2×SYBR Premix Ex Taq™ (TaKaRa, Japan) and SOCS2 primer pairs of Table 2 (Exicycler version 2, Bioneer, Republic of Korea). Concurrently, using GAPDH (Glyceraldehyde-3-phosphate dehydrogenase) as a quantitative control, real time PCR was performed with GAPDH primer pair of Table 2. The PCR condition was as follows: denaturation of 10 min at 95° C.; 40 cycles of sec at 95° C., 30 sec at 60° C., and 1 min at 72° C.; and extension of 8 min at 72° C. followed by cooling to room temperature. After analyzing real time PCR data by the comparative CT (2^{-ΔΔCt}) method which normalizes the amount of socs2 expression of each sample to the amount of GAPDH expression, the relative amount of SOCS2 expression in differentiated NK cells at each day to the amount of socs2 expression in primary NK cells was shown as a graph in FIG. 1(a).

Consequently, as shown in FIG. 1(a), it was found that differentiation proceeded from the fact that NK cells expressing CD56 increased as the incubating time passed by. It was found that SOCS2 mRNA expression also increased in proportion to NK cell differentiation. Particularly, it was found that socs2 mRNA expression increased sharply from after 8 days of incubation.

TABLE 3

| Gene | Sense primer | | Antisense primer | |
|---|---|---|---|---|
| SOCS1 | SEQ ID NO: 8 | 5′-agagcttcgactgcctcttc-3′ | SEQ ID NO: 9 | 5′-ctcaggtagtcgcggaggac-3′ |
| SOCS3 | SEQ ID NO: 10 | 5′-gccacctactgaaccctcct-3′ | SEQ ID NO: 11 | 5′-acggtcttccgacagagatg-3′ |

### Example 4

### The Effect of Inhibition of socs2 Gene Expression on NK Cell Differentiation

IL-15 is an essential cytokine for NK cell differentiation. With SOCS2 expression inhibited, the present inventors measured the NK cell differentiation by IL-15 stimulation. Specifically, primary NK cells were transduced with SOCS2 siRNA (Dharmacon, U.S.A., SEQ ID NO.12: 5′-CGACUACUAUGUUCAGAUG-3′) or control siRNA (Dharmacon, U.S.A., SEQ ID NO.13: 5′-UAGCGACUAAACACAUCAAUU-3′) using Amaxa Human CD34 Cell Nucleofector™ Kit(program U-08), and then, deprivation of IL-15 followed by IL-15 stimulation for 16 hr was carried out by the same method in Example <2-2>. Then, SOCS2 protein expression in the cells was identified by performing Western blot analysis with the same method in Example <2-1>. Concurrently, β-actin expression was also observed by using anti-β-actin primary antibody (Santa Cruz, U.S.A.) as a quantitative control.

Also, to determine whether the inhibition of SOCS2 expression affected the NK cell differentiation or not, primary NK cells were transduced with siRNA and CD-56 expression was measured by performing FACS on the cells at 2, 3, and 5th day.

Consequently, as shown in FIG. 4, SOCS2 protein expression was inhibited by SOCS2 siRNA in primary NK cells and the inhibition of SOCS2 protein expression was not restored by IL-15 stimulation. Also, IL-15 stimulation increased CD-56 expression similarly regardless of whether SOCS2 protein was expressed or not. This suggested that SOCS2 does not affect NK cell differentiation.

### Example 5

### The Effect of Inhibition of socs2 Gene Expression on NK Receptor Signal Transduction

SOCS2 is a member of the SOCS family, which is known to act as negative feedback regulators in cytokine receptor-mediated signaling pathways. STAT5 which is phosphorylated by IL-15 activates JAK/STAT signaling pathway and subsequently regulates gene expression. To assess whether the upregulated SOCS2 by IL-15 acts as a negative feedback regulator in IL-15 signaling, the present inventors inhibited SOCS2 expression in NK cells, and then, examined the STAT5 phosphorylation in the NK cells following IL-15 stimulation. Specifically, NK-92 cells were infected with 10 MOI of either SOCS2 shRNA lentivirus or control shRNA lentivirus prepared in Example <2> and STAT5, phosphorylated STAT5, and SOCS2 protein expression were measured from each shRNA-treated NK-92 cells incubating with or without an IL-15 containing medium, or with a IL-15 containing medium for 10 min following deprivation of IL-15 by performing Western blot analysis with the same method in Example <2-1>. Anti-phosphorylated STAT5 primary antibody (Santa Cruz, U.S.A.) and anti-STAR5 primary antibody (Santa Cruz, U.S.A.) as well as the antibody used in Example <2-1> were additionally used and concurrently, the β-actin expression was observed as a quantitative control.

Consequently, as shown in FIG. 5, regardless of whether SOCS2 protein was expressed or not, phosphorylated STAT5 was observed from all NK-92 cells except for lentivirus-treated NK-92 cells cultured in a medium without IL-15. That the inhibition of SOSC2 expression does not affect STAT5 phosphorylation suggested that SOSC2 does not act as a negative regulator in IL-15 signaling.

### Example 6

### The Effect of Inhibition of socs2 Gene Expression on NK Cell Proliferation

IL-15 is an essential cytokine for NK cell proliferation. With SOCS2 expression inhibited, the present inventors measured the NK cell proliferation by IL-15 stimulation. Specifically, NK-92 cells were infected with 10 MOI of either SOCS2 shRNA lentivirus or control shRNA lentivirus prepared in Example <2> and stimulated with IL-15 for 16 hr using the same method in Example <2-2>. Then, apoptosis was measured by performing FACS on the cells using FITC Annexin V Apoptosis Detection Kit I (BD Pharmingen, U.S.A.).

Consequently, as shown in FIG. 6, regardless of whether SOCS2 protein was expressed or not, cell proliferation was observed from all NK-92 cells. From this, it was found that SOCS2 does not affect the NK cell proliferation.

### Example 7

### The Effect of Inhibition of socs2 Gene Expression on NK Cell Survival

IL-15 is an essential cytokine for NK cell survival. With SOCS2 expression inhibited, the present inventors measured the NK cell survival by IL-15 stimulation. Specifically, NK-92 cells were infected with 10 MOI of either SOCS2 shRNA lentivirus or control shRNA lentivirus prepared in Example <2> and stimulated with IL-15 for 16 hr using the same method in Example <2-2>. Then, to compare perforin, granzyme B, NKp30, NKp40, NKp46, IL-18R, and NKG2D expression in the cells, FACS was performed using anti-perforin antibody, anti-granzyme B antibody, anti-NKp30 antibody, anti-NKp40 antibody, anti-NKp46 antibody, anti-IL-12Rβ antibody, anti-IL-18R antibody, and anti-NKG2D antibody. For the quantitative comparison, IgG expression was measured from each experimental group through FACS.

Consequently, as shown in FIG. 7, regardless of whether SOCS2 protein was expressed or not, perforin, granzyme B, NKp30, NKp40, NKp46, IL-12Rβ, IL-18R, and NKG2D expression were observed similarly from all NK-92 cells. From this, it was found that SOCS2 does not affect the NK cell survival.

### Example 8

### Determination of the Effect of Inhibition of socs2 Gene Expression on NK Cell Activity

<8-1> The Reduction in NK Cell Cytotoxicity by the Inhibition of socs2 Gene Expression

To investigate the effect of SOCS2 of which expression increases by IL-15 on NK cell activity, the cytotoxicity of NK cells in which expression of SOCS2 was silenced was examined. Specifically, NK-92 cells or mature NK cells were infected with 10 MOI of either SOCS2 shRNA lentivirus or control shRNA lentivirus prepared in Example <2> and stimulated with IL-15 for 16 hr following deprivation using the same method in Example <2-2>. K562, Jurkat, MCF7, or A549 cancer cell lines were labeled with 100 μCi at 37° C. for 1 hr and washed three times with PBS. The ability of NK cells to kill tumor cells (cytotoxicity) was measured by a standard ⁵¹Cr-release assay. The IL-15-stimulated NK-92 cells were cultured by limiting dilution, and then, the cells along with 1×10⁴/100 μL of each ⁵¹Cr-labelled cancer cells were added to total 200 μL to a 96-well plate (Corning, U.S.A.) and cultured at 37° C., CO₂ incubator for 4 hr. Then, ⁵¹Cr which was released from tumor cells lysed with NK-92 cells to supernatants was measured using a γ-counter and specific cytotoxicity was calculated using the following Equation 1:

[Equation 1]

Specific cytotoxicity (%)=[(experimental release spontaneous release)/(maximum release−spontaneous release)]×100.

Consequently, as shown in FIG. 8(a), NK-92 cells treated with SOCS2 shRNA had a reduced cytotoxicity against all tumor cells compared to NK-92 cells treated with control shRNA. As shown in FIG. 8(b), mature NK cells treated with SOCS2 shRNA had a reduced cytotoxicity against all tumor cells compared to mature NK cells treated with control shRNA. This suggested that the increase in the NK cell activity by IL-15 resulted from the SOCS2 expression induced by the IL-15.

<8-2> Decrease in IFN-γ Production by NCR Stimulation of NK Cells Due to Inhibition of socs2 Gene Expression

NK cells express NCR which recognizes target cells on their surfaces. Target cell-bound NCR transfers signals into NK cells, and subsequently, NK cells release granzymes and perforin to kill target cells. To investigate the effect of SOCS2 of which expression increases by IL-15 on NK cell activity, the IFN-γ (Interferon-γ) production by NCR stimulation of NK cells in which expression of SOCS2 was silenced was measured by ELISA analysis. Specifically, NK-92 cells were infected with 10 MOI of either SOCS2 shRNA lentivirus or control shRNA lentivirus prepared in Example <2> and stimulated with IL-15 for 16 hr following deprivation using the same method in Example <2-2>. The IL-15-primed NK-92 cells were dispensed into 96-well plates at 3×10⁵ cells/well and cultured without or with NCR stimulation [anti-NKp30 monoclonal antibody (Santa Cruz, U.S.A.), anti-NKp44 monoclonal antibody (Santa Cruz, U.S.A.), or anti-NKp46 monoclonal antibody (Santa Cruz, U.S.A.)] or cytokine stimulation [IL-12 (10 ng/mL) or IL-18 (30 ng/mL)] for 16 hr, and then washed two times with PBS. IFN-γ present in supernatants was measured using human IFN-γ ELISA kit(Assay designs, U.S.A.).

Also, mature NK cells in Example <1-2> were infected with MOI of either SOCS2 shRNA lentivirus or control shRNA lentivirus prepared in Example <2> and stimulated with IL-15 for 16 hr using the same method in Example <2-2>. The IL-15-primed mature NK cells were cultured without or with anti-NKp30 monoclonal antibody (Santa Cruz, U.S.A.), IL-12, or IL-18, and IFN-γ was measured using the above method.

Consequently, as shown in FIG. 9(a), IFN-γ production in NK-92 cells which were treated with SOCS2 shRNA and cultured with NCR stimulation was reduced compared to NK-92 cells treated with control shRNA. However, NK-92 cells cultured with cytokine stimulation did not show the difference in IFN-γ production depending on SOCS2 expression. As shown in FIG. 9(b), IFN-γ production in mature NK cells which were treated with SOCS2 shRNA and cultured with NKp30 stimulation was reduced compared to mature NK cells treated with control shRNA. However, mature NK cells cultured with IL-12 or IL-18 stimulation did not show the difference in IFN-γ production depending on SOCS2 expression. This suggested that SOCS2 increases IFN-γ production of NK cells by NCR stimulation.

<8-3> Decrease in IFN-γ mRNA Expression by NCR Stimulation of NK Cells Due to Inhibition of socs2 Gene Expression

To observe whether the decrease in IFN-γ production by inhibition of SOCS2 expression identified in Example <8-2> results from inhibition of IFN-γ mRNA generation, IFN-γ mRNA expression was examined by performing real time PCR using the same method in Example <2-1> with NK-92 cells treated identically with Example <8-2>. IFN-γ sense primer (SEQ ID NO. 14: 5′-gtccaacgcaaagcaataca-3′) and IFN-γ antisense primer (SEQ ID NO.15: 5′-ctcttcgacctcgaaacagc-3′) were used for IFN-γ amplification.

Consequently, as shown in FIG. 10, IFN-γ mRNA generation in NK-92 cells which were treated with SOCS2 shRNA and cultured with NCR stimulation was reduced compared to NK-92 cells treated with control shRNA. However, NK-92 cells cultured with cytokine stimulation did not show the difference in IFN-γ mRNA generation depending on SOCS2 expression. This suggested that the decrease in IFN-γ production by inhibition of SOCS2 expression results from inhibition of IFN-γ mRNA generation.

### Example 9

### The Effect of Inhibition of socs2 Gene Expression on NK Cell Receptor Signaling Pathways

When a NK cell contact with a target tumor cell, various ligands of the target tumor cell and receptors on the surface of the NK cell interact to proceed various signaling pathways, and subsequently, the NK cell secretes perforin and granzymes, and has the cytotoxic effect against the target tumor cell. To examine the effect of inhibition of SOCS2 expression on NK cell receptor signaling pathways, the following experiment was carried out. Specifically, NK-92 cells were infected with 10 MOI of either SOCS2 shRNA lentivirus or control shRNA lentivirus prepared in Example <2> and stimulated with IL-15 for 16 hr using the same method in Example <2-2>. From NK-92 cells alone or NK-92 cells incubated with target tumor cells, K562 cells for 15 or 30 min, phosphorylation of Src (sarcoma, proto-oncogenic tyrosine kinases) and Syk (Spleen tyrosine kinase) which are involved in NK cell proximal receptor signaling, and phosphorylation of MAPKs [Mitogen-activated protein (MAP) kinases]: JNK (c-Jun N-terminal kinases), ERK (Extracellular signal-regulated kinases), and p38 were measured by performing Western blot analysis with the same method in Example <2-1>. Concurrently, SOCS2 expression and β-actin expression as a quantitative control were measured. Anti-phosphorylated Src primary antibody (Santa Cruz, U.S.A.), anti-phosphorylated Syk primary antibody (Santa Cruz, U.S.A.), anti-phosphorylated JNK primary antibody (Santa Cruz, U.S.A.), anti-phosphorylated ERK primary antibody (Santa Cruz, U.S.A.), anti-phosphorylated p38 primary antibody (Santa Cruz, U.S.A.), Src primary antibody (Santa Cruz, U.S.A.), anti-Syk primary antibody (Santa Cruz, U.S.A.), anti-JNK primary antibody (Santa Cruz, U.S.A.), anti-ERK primary antibody (Santa Cruz, U.S.A.) and anti-p38 primary antibody (Santa Cruz, U.S.A.) as well as the antibody used in Example <2-1> were additionally used.

Consequently, as shown in FIG. 11, phosphorylation of Src and Syk and phosphorylation of JNK among MAPKs were reduced in NK-92 cells in which SOCS2 expression was inhibited. However, ERK and p38 were phosphorylated similarly regardless of whether SOCS2 protein was expressed or not.

### Example 10

### The Effect of Inhibition of socs2 Gene Expression on NK Cell MAPK Signaling Pathways

<10-1> The Effect of SOCS2 on NK Cell MAPK Signaling Pathways in Response to NCR Stimulation

To examine the effect of inhibition of SOCS2 expression on NK cell MAPK signaling pathways in response to NCR stimulation, the following experiment was carried out. Specifically, NK-92 cells were infected with 10 MOI of either SOCS2 shRNA lentivirus or control shRNA lentivirus prepared in Example <2> and stimulated with IL-15 for 16 hr using the same method in Example <2-2>. The IL-15 primed NK-92 cells were culture without or with anti-NKp30 monoclonal antibody for 5, 15, or 30 min. Phosphorylation of JNK, ERK, and p-38 were measured by performing Western blot analysis with the same method in Example <2-1> using anti-phosphorylated JNK primary antibody, anti-phosphorylated ERK primary antibody, anti-phosphorylated p38 primary antibody, anti-JNK primary antibody, anti-ERK primary antibody, and anti-p38 primary antibody. Concurrently, phosphorylation of Src was measured using anti-phosphorylated Src primary antibody and Src primary antibody, and SOCS2 expression and β-actin expression as a quantitative control were measured using anti-SOCS2 primary antibody and anti-β-actin primary antibody.

Consequently, as shown in FIG. 12, phosphorylation of JNK only among MAPKs was reduced in NK-92 cells in which SOCS2 expression was inhibited as NRC stimulation time increased. Phosphorylation of Src was reduced in NK-92 cells in which SOCS2 expression was inhibited in response to NCR stimulation as in Example <9>.

<10-2> Determination of MAPK Affected by SOCS2 During NK Cell Signaling Pathway in Response to NCR Stimulation

Results that phosphorylation of MAPK is related with NK cell activity have already been reported (Vivier et al., Science, 306:1517-1519, 2004). To investigate which kinase among JNK, ERK, and p38 plays the most important role in the following NK cell activities, NK-92 cells were treated with 10 mM of a JNK inhibitor (SP600125), an ERK inhibitor (PD98059), or a p38 inhibitor (SB203580), and cytotoxicity against the K562 cancer cell line by NK cells was measured by the method in Example <8-1> and the NK cell ability to produce IFN-γ in response to NRC stimulation was measured by the method in Example <8-2>.

Consequently, as shown in FIG. 13(a) and FIG. 13(b), NK-cells treated with a JNK inhibitor had the most significantly reduced cytotoxicity and IFN-γ production. This result suggested that the reduced JNK phosphorylation of NK-92 cells in which SOCS2 expression was inhibited, which was confirmed in Example <10-1> was a major cause of the decreased cell activity of SOCS2-inhibited NK cells.

### Example 11

### Preparation of SOCS2 Expression Vectors and Pyk2 Expression Vectors

<11-1> Preparation of a SOCS2 Expression Vector

The cDNA encoding SOCS2 was obtained from Mammalian Gene Collection (NIH, USA) and amplified by PCR using Pfu polymerase (Stratagene, U.S.A.) and the PCR product was inserted into the BamHI and ClaI restriction enzyme sites of the pEBG vector (AddGene, U.S.A.). A GST tag was added to the N-terminal of SOCS2. Specifically, the forward primer of SEQ ID NO:16 (5′-GGATCCATGACCCTGCGGTGCCTTGAGCCCTCCGGGAATGGCGGGG-3′) and the reverse primer of SEQ ID NO:17 (5′-ATCGATTTATACCTGGAATTTATATTCTTCCAAGTAATCTTTTAGTC-3′) were used for PCR amplification of the socs2 gene. The PCR condition was as follows: SOCS2 cDNA was used as a template; 94° C. for 4 min, 25 cycles of 94° C. for 30 sec, 58° C. for 30 sec, 72° C. for 4 min, and extension of 72° C. for 10 min. The amplified PCR product and pEBG were cut with BamHI and ClaI and purified. About 100 ng of the vector, about 100 ng of the insert fragment, and 1 unit of T4 ligase (Roche, Switzerland) were mixed to incubate at 16° C. for 16 hr. After ligation, the resulting vector was transformed into E. coli DH5 (Invitrogen, U.S.A.) and selected from LB agar plates containing ampicillin. A plasmid having a DNA fragment of interest was obtained by a suitable restriction enzyme, and identified finally through DNA sequencing. The prepared expression vector was named ‘pGST-SOCS2’.

<11-2> Preparation of a Pyk2 Expression Vector

The cDNA encoding Pyk2 (protein tyrosine kinase 2) was obtained from Mammalian Gene Collection (NIH, USA) and amplified by PCR using Pfu polymerase (Stratagene, U.S.A.) and the PCR product was inserted into the EcoRI and SalI restriction enzyme sites of the pBICEP-CMV-1 vector (Sigma). A Flag tag was added to the N-terminal of Pyk2. Specifically, the forward primer of SEQ ID NO:19 (5′-GAATTCGATGTCTGGGGTGTCCGAGCCCCTGAGTCGAGTAAAGTTGGG-3′) and the reverse primer of SEQ ID NO:20 (5′-GTCGACTCACTCTGCAGGTGGGTGGGCCAGATTGGCCAGAACCTTGGC-3′) were used for PCR amplification of the Pyk2 gene. The PCR condition was as follows: Pyk2 cDNA was used as a template; 94° C. for 4 min, 25 cycles of 94° C. for 30 sec, 58° C. for 30 sec, 72° C. for 4 min, and extension of 72° C. for 10 min. The amplified PCR product and pBICEP-CMV-1 were cut with EcoRI and SalI and purified. About 100 ng of the vector, about 100 ng of the insert fragment, and 1 unit of T4 ligase (Roche, Switzerland) were mixed to incubate at 16° C. for 16 hr. After ligation, the resulting vector was transformed into E. coli DH5 (Invitrogen, U.S.A.) and selected from LB agar plates containing ampicillin. A plasmid having a DNA fragment of interest was obtained by a suitable restriction enzyme, and identified finally through DNA sequencing. The prepared expression vector was named ‘pFlag-Pyk2’.

<11-3> Preparation of Mutant SOCS2 Expression Vectors

For use in the experiment to determine which region of SOCS2 interacted with Pyk2, SOCS2 deletion mutants were prepared. The cDNA encoding SOCS2 was obtained from Mammalian Gene Collection (NIH, USA) and amplified by PCR using Pfu polymerase (Stratagene, U.S.A.) and the PCR product was inserted into the BamHI and ClaI restriction enzyme sites of the pEBG vector (AddGene, U.S.A.). In order to enhance the purification efficiency, a GST tag was added to the N-terminal of SOCS2. Specifically, the forward primer of SEQ ID NO:23 (5′-GGATCCATGACCCTGCGGTGCCTTGAGCCCTCCGGGAATGGCGGGG-3′) and the reverse primer of SEQ ID NO:24 (5′-ATCGATTTACTGACCGAGCTCCCGCAGGGCCTTCGCCAGACGCG-3′) were used for PCR amplification of the mutant socs2 (SEQ ID NO:22) in which SH2 (Src homology 2) domain (SEQ ID NO:21) was deleted. the forward primer of SEQ ID NO:26 (5′-GGATCCATGACCCTGCGGTGCCTTGAGCCCTCCGGGAATGGCGGGG-3′) and the reverse primer of SEQ ID NO:27 (5′-ATCGATTTAAAGGTGAACAGTGCCGTTCCGGGGGGCTTCTGGACC-3′) were used for PCR amplification of the mutant socs2 (SEQ ID NO:25) in which SOCS domain was deleted. The PCR condition was as follows: SOCS2 cDNA was used as a template; 94° C. for 4 min, 25 cycles of 94° C. for 30 sec, 58° C. for 30 sec, 72° C. for 4 min, and extension of 72° C. for 10 min. The each amplified PCR product and pEBG were cut with BamHI and ClaI and purified. About 100 ng of the vector, about 100 ng of the insert fragment, and 1 unit of T4 ligase (Roche, Switzerland) were mixed to incubate at 16° C. for 16 hr. After ligation, the resulting vector was transformed into E. coli DH5 (Invitrogen, U.S.A.) and selected from LB agar plates containing ampicillin. A plasmid having a DNA fragment of interest was obtained by a suitable restriction enzyme, and identified finally through DNA sequencing. The prepared expression vectors were named ‘pGST-SOCS2-ΔSH2’ and ‘pGST-SOcS2-ΔSOCS’, respectively.

<11-4> Preparation of a Mutant Pyk2 Expression Vector

In order to prepare an expression vector of a mutant Pyk2 in which tyrosine 402 had been mutated to phenylalanine, pFlag-Pyk2 vector prepared in Example <11-2> and QuickChange Site-Directed Mutagenesis kit (Stratagene, U.S.A.) were used for the preparation of the mutant Pyk2 expression vector. The mutant Pyk2 expression vector was prepared according to manufacturer's instructions. The primer of SEQ ID NO:28 (5′-CAGCATAGAGTCAGACATCTTCGCAGAGATTCCCGACGAAAC-3′) was used. The mutant Pyk expression vector was named ‘pFlag-Pyk2-Y402F’.

### Example 12

### Verification of Interaction Between SOCS2 and Pyk2

To verify the binding between Pyk2 protein and SOCS2 protein which were identified by a yeast two-hybrid screening (dualsystems Biotech, Switzerland) from a human-derived cell line, the following experiments were carried out using the expression vectors obtained in Examples <11-1> to <11-4>.

<12-1> Determination of the Binding Between SOCS2 and Pyk2 in a Human Cell Line

The GST expression vector and pFlag-Pyk2, or pGST-SOCS2 and pFlag-Pyk2 were cotransformed into 293T cells using Lipofectamin 2000 (Invitrogen, U.S.A.). After 4 hr of transformation, the medium was replaced to a general culture medium or MG132 (proteasome inhibitor)-containing medium, and hr later, cells were lysed. The each cell lysate was allowed to react with glutathione-sepharose bead (GE Healthcare, U.S.A.) at 4° C. for 4 hr. Western blot analysis was performed by the same method in Example <2-1> using anti-Flag primary antibody (Santa Cruz, U.S.A.) or anti-GST primary antibody (Santa Cruz, U.S.A.).

Consequently, as shown in FIG. 15(a), Flag was detected only in cells cotransformed with pGST-SOCS2 and pFlag-Pyk2. Cells treated with MG132 showed a significantly large Flag band compared to cells which were not treated with MG132.

Therefore, it was found that SOCS2 interacted with Pyk2 to coprecipitate in a human cell line like the result of the yeast two-hybrid experiment, and Flag-tagged Pyk2 was degraded by proteasome.

<12-2> Determination of the Effect of SOCS2 on Pyk2 Degradation in a Human Cell Line

In order to determine whether SOCS2 inhibits the proteasome-mediated Pyk2 degradation or not, the following experiment was carried out. First of all, pGST-SOCS2, pFlag-Pyk2, and HA-ubiquitin (AddGene) were cotransformed into 293T cells using Lipofectamin 2000. After 4 hr of transformation, the medium was replaced to a general culture medium or MG132 (proteasome inhibitor)-containing medium, and 24 hr later, cells were lysed. The each cell lysate was allowed to react with anti-Flag antibody or anti-GST antibody. The antigen-antibody complexes were precipitated by incubation at 4° C. for 1 day with G-protein-conjugated agarose (Roshe, Switzerland). The immunoprecipitated complexes were washed with 1×PBS. Western blot analysis was performed by the same method in Example <2-1> using anti-GST antibody, anti-Flag antibody, or anti-HA antibody (Santa Cruz, U.S.A.).

Consequently, as shown in FIG. 15(b), GST and HA were detected only in the cell lysate precipitated with anti-Flag antibody. Cells treated with MG132 showed a significantly large GST band and more HA bands compared to cells which were not treated with MG132. This indicated that SOCS2 interacted with Pyk2, and then, induced the ubiquitination of Pyk2.

<12-3> Determination of SOCS2 Domain Interacting with Pyk2 in 293T Cells

To determine a SOCS2 domain which is important in the binding with Pyk2, the present inventors carried out the following experiment. HA-Ubiquitin, pFlag-Pyk2, and, GST expression vector, pGST-SOCS2, pGST-SOCS2-ΔSH2 or pGST-SOCS2-ΔSOCS were cotransformed into 293T cells using Lipofectamin 2000. After 4 hr of transformation, the medium was replaced to a new culture medium, and 24 hr later, cells were lysed. The each cell lysate was allowed to react with anti-Flag antibody. The antigen-antibody complexes were precipitated by incubation at 4° C. for 1 day with G-protein-conjugated agarose. The immunoprecipitated complexes were washed with 1×PBS. Western blot analysis was performed by the same method in Example <2-1> using anti-GST antibody, anti-Flag antibody, or anti-HA antibody (Santa Cruz, U.S.A.).

Consequently, as shown in FIG. 16(a), the interaction between SOCS2 and Pyk2 were shown in cells transformed with pGST-SOCS2 and cells transformed with pGST-SOCS2-ΔSOCS, but not shown in cells transformed with pGST-SOCS2-ΔSH2. This indicated that SH domain of SOCS2 was important in the SOCS2 binding with Pyk2.

<12-4> Determination of the Binding Between SOCS2 and Pyk2 in NK Cells

To determine whether the binding between SOCS2 and Pyk2 determined in 293T cells also occurs between SOCS2 and Pyk2 which were expressed endogenously in NK cells, the following endogenous immunoprecipitation experiment was carried out. NK-92 cells were lysed and the cell lysates were allowed to react with anti-IgG antibody or anti-SOCS2 antibody. The antigen-antibody complexes were precipitated by incubation at 4° C. for day with G-protein-conjugated agarose. The immunoprecipitated complexes were washed with 1×PBS. Western blot analysis was performed by the same method in Example <2-1> using anti-Pyk2 antibody, anti-SOCS2 antibody and anti-phosphorylated Pyk2 antibody [antibody which recognizes p-Pyk2^{Tyr402}, Pyk2 in which tyrosine 402 was phosphorylated] (Santa Cruz, U.S.A.).

Consequently, as shown in FIG. 16(b), it was confirmed that SOCS2 which was expressed endogenously also in NK cells interacted with Pyk2 and p-Pyk2^{Tyr402}.

<12-5> Determination of the Binding Between SOCS2 and p-Pyk2^{Tyr402} in NK Cells

From Example <12-4>, it was confirmed that phosphorylated Pyk2 and SOCS2 bind each other. To determine whether phosphorylation of Pyk2 is important in the binding with SOCS2 or not, the present inventors carried out the following experiment. First of all, HA-ubiquitin, pGST-SOCS2, and, pFlag-Pyk2 or pFlag-Pyk2-Y402F were cotransformed into NK-92 cells using Lipofectamin 2000. After 4 hr of transformation, the medium was replaced to a new culture medium, and 24 hr later, cells were lysed. The each cell lysate was allowed to react with anti-Flag antibody. The antigen-antibody complexes were precipitated by incubation at 4° C. for 1 day with G-protein-conjugated agarose. The immunoprecipitated complexes were washed with 1×PBS. Western blot analysis was performed by the same method in Example <2-1> using anti-p-Pyk2^{Tyr402} antibody, anti-GST antibody, anti-Flag antibody or anti-HA antibody.

Consequently, as shown in FIG. 17, the interaction between SOCS2 and Pyk2 were shown in cells transformed with pFlag-Pyk2, but not shown in cells transformed with pFlag-Pyk2-Y402F. This indicated that phosphorylation of Pyk2 was important in the Pyk2 binding with SOCS2.

### Example 13

### Regulation of p-Pyk2^{Tyr402} with Increased SOCS2 by IL-15

To assess how SOCS2 which binds with p-Pyk2^{Tyr402} in NK cells regulates Pyk2, the following experiment was carried out. With NK-92 cells cultured without or with IL-15 for 6, 12 or 24 hr using the same method in Example <2-2>, Western blot analysis was carried out using the same method in Example <2-1> with anti-SOCS2 antibody, anti-p-Pyk2^{Tyr402} antibody, anti-Pyk2 antibody and anti-GAPDH antibody. Moreover, with primary

NK cells cultured without or with IL-15, Western blot analysis was carried out using the same method in Example <2-1> with anti-SOCS2 antibody, anti-p-Pyk2^{Tyr402} antibody, anti-Pyk2 antibody and anti-GAPDH antibody.

Consequently, as shown in FIG. 18(a), SOCS2 expression increased in an IL-15 stimulation time-dependent manner in NK-92 cells and concurrently, phosphorylation of tyrosine 402 in Pyk2 decreased. In addition, as shown in FIG. 18(b), IL-15 stimulation induced the expression of SOCS2 and the decrease in the phosphorylation of Pyk2.

### Example 14

### Determination of the Ubiquitin-mediated Proteasomal Degradation of p-Pyk2^{Tyr402} by SOCS2

<14-1> Determination of the Ubiquitin-mediated Proteasomal Degradation of p-Pyk2^{Tyr402} by SOCS2

To determine whether the decrease in p-Pyk2^{Tyr402} by SOCS2 expression, confirmed in Example <13> resulted from the ubiquitin-mediated proteasomal degradation of p-Pyk2^{Tyr402} by increased SOCS2 by IL-15, the following experiment was carried out. NK-92 cells stimulated with IL-15 as in Example <2-2> were lysed and the cell lysates were allowed to react with anti-Pyk2 antibody. The antigen-antibody complexes were precipitated by incubation at 4° C. for 1 day with G-protein-conjugated agarose. The immunoprecipitated complexes were washed with 1×PBS. Western blot analysis was performed by the same method in Example <2-1> using anti-ubiquitin antibody (Santa Cruz, U.S.A.), anti-SOCS2 antibody, anti-p-Pyk2^{Tyr402} antibody, anti-Pyk2 antibody, and anti-GAPDH antibody.

Consequently, as shown in FIG. 19, as the expression of SOCS2 was induced by IL-15 stimulation in NK-92 cells, phosphorylation of Pyk2 decreased and ubiquitination of Pyk2 increased. This indicated that SOCS2 protein induced the ubiquitin-mediated proteasomal degradation of p-Pyk2^{Tyr402}.

<14-2> Determination of Inhibition of Pyk2 Degradation by Inhibiting SOCS2 Expression

With NK-92 cells treated with SOCS2 shRNA or control shRNA by the same method in Example <2-2> and mature NK cells treated with SOCS2 siRNA or control siRNA, Western blot analysis was performed by the same method in Example <2-1> using anti-SOCS2 antibody, anti-p-Pyk2^{Tyr402} antibody, anti-Pyk2 antibody, and anti-β-actin antibody.

Consequently, as shown in FIG. 20, both the p-Pyk2^{Tyr402} expression and Pyk2 expression increased when SOCS2 expression was inhibited in NK-92 cells and mature NK cells, compared to NK-92 cells and mature NK cells in which SOCS2 was expressed. It was considered that this resulted from that p-Pyk2^{Tyr402} and Pyk2 were not degraded due to inhibition of SOCS2 expression.

### Example 15

### Determination of the Effect of SOCS2-induced Inhibition of Pyk2 Expression on the NK Cell Activity

To assess whether the reduced NK cell activity when SOCS2 expression was inhibited resulted from the inhibition of p-Pyk2^{Tyr402} degradation by SOCS2, the following experiment was carried out.

<15-1> Preparation of GFP-Pyk2 Expression Vector and Overexpression of Pyk2

The cDNA (SEQ ID NO:18) encoding Pyk2 was obtained from Mammalian Gene Collection (NIH, USA) and amplified by PCR using Pfu polymerase (Stratagene, U.S.A.) and the PCR product was inserted into the XhoI and EcoRI restriction enzyme sites of the pLVX-AcGFP-C1 vector (Clontech, U.S.A.). A GFP tag was added to the N-terminal of Pyk2. Specifically, the forward primer of SEQ ID NO:29 (5′-CTCGAGCCATGTCTGGGGTGTCCGAGCCCCTGAGTCGAGTAAAGTTG-3′) and the reverse primer of SEQ ID NO:30 (5′-GAATTCTCACTcTGCAGGTGGGTGGGCCAGATTGGCCAGAACCTTGGC-3′) were used for PCR amplification of the Pyk2 gene. The PCR condition was as follows: Pyk2 cDNA was used as a template; 94° C. for 4 min, 25 cycles of 94° C. for 30 sec, 58° C. for 30 sec, 72° C. for 4 min, and extension of 72° C. for 10 min. The each amplified PCR product and pLVX-AcGFP-C1 were cut with XhoI and EcoRI and purified. About 100 ng of the vector, about 100 ng of the insert fragment, and 1 unit of T4 ligase (Roche, Switzerland) were mixed to incubate at 16° C. for 16 hr. After ligation, the resulting vector was transformed into E. coli DH5 (Invitrogen, U.S.A.) and selected from LB agar plates containing ampicillin. A plasmid having a DNA fragment of interest was obtained by a suitable restriction enzyme, and identified finally through DNA sequencing. The prepared expression vector was named ‘pGFP-Pyk2’.

To reproduce the situation in which the regulation of p-Pyk2^{Tyr402} mediated by SOCS2 was broken, the pGFP-Pyk2 expression vector prepared in Example <15-1> was transformed into NK-92 cells using Lipofectamin. Western blot analysis was performed by the same method in Example <2-1> using anti-GFP antibody, anti-SOCS2 antibody, anti-p-Pyk2^{Tyr402} antibody, anti-Pyk2 antibody and anti-β-actin antibody.

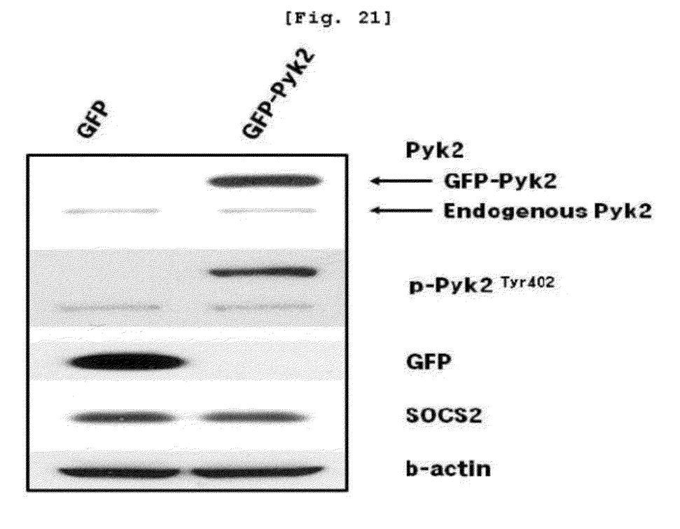

Consequently, as shown in FIG. 21, even though SOCS2 was overexpressed in cells which overexpress exogenous Pyk2, almost endogenous Pyk2 disappeared, but exogenous Pyk2 and p-Pyk2^{Tyr402} were overexpressed.

<15-2>Determination of the Effect of the Increase in Pyk2 Protein on the NK Cell Activity

The present inventors examined the cytotoxicity against the K562 cancer cell line and the ability to produce IFN-γ in response to NRC stimulation of NK-92 cells in which Pyk2 was overexpressed were measured by the method in Example <8-1> and the method in Example <8-2>, respectively.

Consequently, as shown in FIG. 22(a) and FIG. 22(b), the cytotoxicity and IFN-γ production were reduced the most, when Pyk2 was overexpressed in NK-92 cells. This result indicated that the reason why the NK cell activity was reduced when SOCS2 was inhibited was that the inhibition of SOCS2 expression broke the regulation of p-Pyk2^{Tyr402}.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1-2. (canceled)

3. A method for activating natural killer cells comprising treating natural killer cells with SOCS2 protein comprising an SH2 (Src homology 2) domain encoded by a nucleic acid molecule having polynucleotide sequence of SEQ ID NO:21.

4. The method as set forth in claim 3, wherein the SOCS2 protein comprising an SH2 domain is encoded by the polynucleotide of SEQ ID NO:1.

5. The method as set forth in claim 4, wherein the SOCS2 protein comprising an SH2 domain has the amino acid sequence set forth as SEQ ID NO:25.

6. The method as set forth in claim 3, further comprising examining whether the natural killer cells were activated or not.

7. The method as set forth in claim 6, wherein whether the natural killer cells were activated or not is determined by:
i) examining in comparison with a control group whether the expression of Pyk2 protein decreased or not;
ii) examining whether IFN-γ production of experimental group increased or not compared to control when NCR (natural cytotoxicity receptor) stimulation was given to cells; or
iii) examining whether the ability to kill target cells of experimental group increased or not compared to control.

8. A method for activating natural killer cells comprising the steps of:
(1) preparing an expression vector wherein the socs2 gene having polynucleotide sequence of SEQ ID NO:1 is operably linked; and
(2) transducing the expression vector prepared in step (1) into natural killer cells.

9-10. (canceled)

11. A method for screening SOCS2 having the increased natural killer cell-activating effect, comprising the steps of:
(1) preparing a first expression vector wherein the Pyk2 gene having polynucleotide sequence of SEQ ID NO:18 is operably linked;
(2) preparing second expression vectors wherein a polynucleotide is operably linked, the polynucleotide encoding a mutant SOCS2 in which a SH2 domain encoded by the polynucleotide of SEQ ID NO:21 is conserved and a mutation occurred at the polynucleotide sequence excluding the SH2 domain within SOCS2;
(3) transducing the first expression vector in step (1) and each the second expression vector in step (2), together or one after another, into natural killer cells; and
(4) determining whether the activity of transduced natural killer cells in step (3) increased or not compared to control.

12. The method as set forth in claim 11 wherein the SOCS2 in step (2) is encoded by the polynucleotide of SEQ ID NO:1.

13. The method as set forth in claim 11, wherein the mutation in step (2) occurs by substitution, addition, or deletion of one or more amino acid residues.

14. The method as set forth in claim 11, wherein the mutant SOCS2 in step (2) is encoded by the polynucleotide of SEQ ID NO:25

15. The method as set forth in claim 11, wherein whether the activity of natural killer cells increased or not is determined by:
i) examining in comparison with a control group whether the expression of Pyk2 protein decreased or not;
ii) examining whether IFN-γ production of experimental group increased or not compared to control when NCR (natural cytotoxicity receptor) stimulation was given to cells; or
iii) examining whether the ability to kill target cells of experimental group increased or not compared to control.

16-19. (canceled)
